(19) United States
(12) Patent Application Publication
(10) Pub. No.: US 2024/0084241 A1
(43) Pub. Date: Mar. 14, 2024

(54) PREDICTION OF DISTRIBUTION OF GLYCANS ATTACHED TO MOLECULES MANUFACTURED IN A CELL CULTURE

(71) Applicant: GENENTECH, INC., South San Francisco, CA (US)

(72) Inventors: Arthi NARAYANAN, San Jose, CA (US); Aditya Avdhut WALVEKAR, Santa Clara, CA (US); Georo L. ZHOU, Richmond, CA (US); Nicholas RUMMEL, San Francisco, CA (US); Zheng LI, South San Francisco, CA (US); Steven J. MEIER, Burlingame, CA (US)

(73) Assignee: GENENTECH, INC., South San Francisco, CA (US)

(21) Appl. No.: 18/464,135

(22) Filed: Sep. 8, 2023

Related U.S. Application Data
(60) Provisional application No. 63/405,350, filed on Sep. 9, 2022.

(51) Int. Cl. C12M 1/36; G16B 40/20

(52) U.S. Cl. CPC C12M 41/48; G16B 40/20

(57) ABSTRACT

A method, system, and non-transitory computer readable medium for predicting a glycan distribution of one or more glycans attached to molecules during a biomolecules manufacturing process are disclosed. In various embodiments, at least three manufacturing process parameters related to the process for manufacturing the molecules are input into a probabilistic graphical model that is trained to predict glycan distribution. The trained probabilistic graphical model may then analyze the at least three manufacturing process parameters to predict the distribution of the glycans that are attached to the molecules.

This application claims priority to U.S. Provisional Patent Application Ser. No. 63/405,350, filed Sep. 9, 2022, which is incorporated by reference herein in its entirety.

### FIELD

Embodiments of this description are generally directed towards predicting distribution of glycans attached to molecules manufactured during a biomolecule manufacturing process. More specifically, this description provides methods and systems for training a probabilistic graphical model, and using the trained probabilistic graphical model, to predict the glycan distribution.

### INTRODUCTION

Glycosylation patterns, patterns of sugar moiety attachments to molecules such as monoclonal antibodies, can be quality attributes of the molecules. This is because glycosylation patterns can affect the stability, solubility, half-life, activities, etc., of the molecules. As such, the quality and effectiveness of a process for manufacturing the molecules can be characterized by monitoring the glycans that are attached to the molecules manufactured via a biomolecule manufacturing process. Sampling the cell culture in which the molecules are manufactured, however, can be time- and resource-intensive as well as a source of product contamination. Accordingly, there is a need for techniques that allow determining or predicting the distribution of glycans attached to molecules during the biomolecules manufacturing process without the need for product sampling.

### SUMMARY

In various embodiments, the disclosure provides systems, compositions, and methods for the analysis of biological samples comprising molecules having one or more glycans attached thereto. In some embodiments, the disclosure provides methods for evaluating a biological production process comprising molecules having one or more glycans attached thereto. In some embodiments, the disclosure provides methods for evaluating a culture medium comprising molecules having one or more glycans attached thereto. In some embodiments, the disclosure provides methods for evaluating a level of one or more culture components in a culture medium comprising molecules having one or more glycans attached thereto.

In various aspects, computer-implemented methods, systems, and compositions are provided for predicting a glycan distribution of one or more glycans attached to molecules manufactured in a cell culture in a bioreactor. In some embodiments, the disclosure encompasses artificial intelligence/machine learning (AI/ML)-powered methods, systems, and compositions for predicting glycan distribution on molecules during a biomanufacturing process by monitoring indicators of glycan distribution on the molecules. In certain embodiments, the computer-implemented method comprises receiving, at a processor, at least three manufacturing process parameters selected from a set of manufacturing process parameters measured from a cell culture in a bioreactor during the biomolecules manufacturing process. In some instances, the set of manufacturing process parameters includes a lactate concentration in the cell culture, total base added into the cell culture during the biomolecules manufacturing process, an osmolality of the cell culture, a viability of the cell culture, a concentration of sodium in the cell culture, specific production of immunoglobulin G in the cell culture, an amount of carbon dioxide sparged into the cell culture, an amount of oxygen sparged into the cell culture; and a packed cell volume of the cell culture, wherein each manufacturing process parameter is listed in order of effect on the glycan distribution. Further, a trained probabilistic graphical model may be used to analyze the at least three manufacturing process parameters and generate the glycan distribution based on the analysis.

In one aspect, there is a computer-implemented method for predicting a glycan distribution of one or more glycans attached to molecules during a biomolecules manufacturing process, the method comprising:
receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution;
generating, via the processor, an indicator of glycan distribution by providing the at least three parameters as input to a probabilistic graphical model that has been trained to predict the glycan distribution using training data comprising, for each of a plurality of cell cultures in a biomolecules manufacturing process, values of the manufacturing process parameters and corresponding measured values of the indicator of glycan distribution.

In various embodiments of the above aspect, the probabilistic graphical model is a Bayesian network model or a probabilistic graphical model is a Markov random field model.

In certain embodiments of the above aspect, the glycan distribution indicates relative proportions of the one or more glycans attached to the molecules, the method further comprising: adjusting at least one of the set of manufacturing process parameters to change the relative proportions of the one or more glycans.

In specific embodiments of the above aspect, the glycans include one or more of Man5, G0F-N, G0-N, G0, G1, G0F, G1F, or G2F.

In certain embodiments of the above aspect, at least one of the set of manufacturing process parameters is measured by a sensor operationally connected to the bioreactor.

In some embodiments of the above aspect, the at least one of the set of manufacturing process parameters is the total volume of the cell culture or the osmolality, and the sensor is a scale configured to weigh the cell culture or an osmometer, respectively, disposed within the bioreactor.

In specific embodiments of the above aspect, at least one of the set of manufacturing process parameters is an output of a controller operationally connected to the bioreactor.

In some embodiments of the above aspect, the one of the set of manufacturing process parameters is the amount of carbon dioxide sparged into the cell culture, or the amount of oxygen sparged into the cell culture, and the controller is an air flow controller configured to control flow of the carbon dioxide sparged into the cell culture or the oxygen sparged into the cell culture, respectively.

In particular embodiments of the above aspect, the molecules include a monoclonal antibody.

One aspect of the disclosure comprises a system for predicting a glycan distribution of one or more glycans attached to molecules during a biomolecules manufacturing process, the system comprising:
a non-transitory memory storing instructions; and
a processor coupled to the non-transitory memory and configured to read the instructions from the non-transitory memory to cause the system to perform operations comprising:
   receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution; and
   analyzing the at least three parameters using a trained probabilistic graphical model to predict the glycan distribution; and
   generating the glycan distribution based on the analyzing.

In specific embodiments of any aspect herein, the probabilistic graphical model is a Bayesian network model or is a Markov random field model.

In various embodiments of any aspect herein, the glycan distribution indicates relative proportions of the one or more glycans attached to the molecules, the operations further comprising: adjusting at least one of the set of manufacturing process parameters to change the relative proportions of the one or more glycans. In specific embodiments of any aspect herein, the glycans include one or more of Man5, G0F-N, G0-N, G0, G1, G0F, G1F, or G2F.

In one aspect of the disclosure, there is a non-transitory computer-readable medium (CRM) having stored thereon computer-readable instructions executable to cause performance of operations for predicting a glycan distribution of one or more glycans attached to molecules during a biomolecules manufacturing process, the operations comprising:
receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution;
analyzing the at least three parameters using a trained probabilistic graphical model to predict the glycan distribution; and
generating the glycan distribution based on the analyzing.

In certain embodiments of any aspect herein, the probabilistic graphical model is a Bayesian network model or is a Markov random field model.

In some embodiments of any aspect herein, the glycan distribution indicates relative proportions of the one or more glycans attached to the molecules, the operations further comprising:
adjusting at least one of the set of manufacturing process parameters to change the relative proportions of the one or more glycans.

In some embodiments of any aspect herein, the glycans include one or more of Man5, G0F-N, G0-N, G0, G1, G0F, G1F, or G2F.

In one aspect of the disclosure, there is a computer-implemented method for predicting a glycan distribution of one or more glycans attached to molecules during a biomolecules manufacturing process, the method comprising:
receiving, at a processor, at least three manufacturing process parameters selected from a set of manufacturing process parameters measured from a cell culture in a bioreactor during the biomolecules manufacturing process, wherein the at least three manufacturing process parameters are selected from the group consisting of lactate concentration per cell culture volume per time; osmolality per time; base total per VCD per time; cell viability per time; sodium concentration per cell culture volume per time; amount of oxygen sparged into the cell culture per VCD per time; amount of carbon dioxide sparged into the cell culture per VCD per time; PCV per time; and qIgG;
generating an indicator of glycan distribution, via the processor, by providing the at least three parameters as input to a probabilistic graphical model that has been trained to predict the glycan distribution using training data comprising, for each of a plurality of cell cultures in a biomolecule manufacturing process, values of the at least three manufacturing process parameters and corresponding measured values of the indicator of glycan distribution

In an embodiment of any aspect herein, the at least three manufacturing process parameters are selected from: the at least three manufacturing process parameters are selected from the group consisting of lactate concentration per cell culture volume per time; osmolality per time; base total per VCD per time; cell viability per time; sodium concentration per cell culture volume per time; amount of oxygen sparged into the cell culture per VCD per time; amount of carbon dioxide sparged into the cell culture per VCD per time; PCV per time; and qIgG.

In various embodiments of any of the preceding aspects, the biomolecule manufacturing process is a process for manufacturing a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid and/or wherein the cells in the cell culture, produce a monoclonal antibody, a complex antibody, an antibody fragment, a virus or a viral particle, a biopharmaceutical, a cytokine, a fusion protein, a growth factor, an immunogenic composition, a vaccine, a lipid, a carbohydrate, and/or a nucleic acid.

The methods or system of any of the present aspects may have any one or more of the features described in relation to any other aspect.

The methods of the present aspect may comprise predicting glycan distribution of one or more glycans attached to biomolecules during a biomolecules manufacturing process in a cell culture, such as in a bioreactor, during a biomolecule manufacturing process, according to any embodiment of any aspect encompassed herein.

In a further aspect, there is provided a computer-implemented method of obtaining a tool for predicting glycan distribution of one or more glycans attached to biomolecules from cells of a cell culture in a bioreactor during a biomolecule manufacturing process, the method comprising:
receiving training data comprising, for each of a plurality of cell cultures in a biomolecule manufacturing process:
values of at least three manufacturing process parameters selected from the set of manufacturing process parameters is listed in Table 1, optionally in order of effect on the glycan distribution; and
corresponding measured values of an indicator of glycan distribution; and
training a machine learning model to generate an indicator of glycan distribution of the cell culture using said training data, the machine learning model taking as input the values of the at least three manufacturing process parameters and providing as output a predicted indicator of glycan distribution of one or more glycans on the biomolecules.

Also described herein according to a further aspect is a system comprising a non-transitory memory storing instructions; and a processor coupled to the non-transitory memory and configured to read the instructions from the non-transitory memory to cause the system to perform any of the methods of the above aspect.

Also described herein according to a further aspect is a non-transitory computer-readable medium (CRM) having stored thereon computer-readable instructions executable that when executed by a processor cause the processor to implement the method of any embodiment of a method of obtaining a tool for predicting glycan distribution of one or more glycans on a biomolecule produced by cells of a cell culture as described herein.

The method of an aspect may further comprise identifying a control action based on the predicted indicator of glycan distribution. The control action may be selected from: stopping the cell culture, measuring product critical quality attributes (CQA) like glycosylation etc., measuring the product titer, harvesting the spent media, changing the value of one or more manufacturing process parameters (for e.g. pH shift, temp shift, adding base, sparging of CO₂ or other gas, etc.), starting or continuing a feed, adding fresh cell culture media and/or cell culture supplements, sending an alert to a computing device or user interface, etc.

Also described herein are methods of monitoring and/or controlling a biomolecule manufacturing process comprising a cell culture in a bioreactor, the methods comprising predicting glycan distribution of one or more glycans on biomolecules produced from cells of the cell culture using a method according to any embodiment of the any aspect herein. The method may further comprise comparing the predicted glycan distribution to one or more reference value. The method may further comprise determining whether the process is operating normally based on the comparison. The method may further comprise issuing an alert when it is determined that the process is not operating normally. The reference value may be an expected glycan distribution or range of glycan distribution, for example obtained from previous processes that are known to have operated normally. A process may be considered to have operated normally if the product of the process met one or more predetermined critical quality attributes (CQAs). The reference value may be a previously predicted glycan distribution for the same process. For example, the comparison may determine whether the glycan distribution has decreased or decreased by more than a predetermined value, compared to a prediction at a previous time point of the process. An alert may be issued when the comparison indicates that the glycan distribution has decreased or decreased by more than a predetermined value, compared to a prediction at a previous time point of the process.

In certain embodiments of the aspects described above, the at least three manufacturing process parameters do not include any manufacturing process parameter that is obtained by analyzing cells sampled from the cell culture.

In other embodiments of the aspects described above, the at least three manufacturing process parameters do not include any manufacturing process parameter that is measured by analyzing a sample of cell culture obtained from the bioreactor.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a glycan distribution prediction system, in accordance with various embodiments.

FIG. 2 is a workflow of a process for training a probabilistic graphical model to predict distribution of glycans attached to biomolecules during a biomolecules manufacturing process, in accordance with various embodiments.

FIG. 3 is a flowchart of a probabilistic model-based process for predicting distribution of glycans attached to biomolecules during a biomolecules manufacturing process, in accordance with various embodiments.

FIG. 4A shows a heatmap or a correlation between MPPs on the X-axis (molecule manufacture process parameters) and glycan targets that are to be predicted (Y-axis). The heatmap is a simplified visual depiction of correlation between features (MPP) and glycan targets, that facilitates the process of selecting features as inputs for the ML (machine learning) model. Features (MPP) with high correlation to the glycan target are selected to be fed as inputs whilst MPP with low correlation are eliminated.

FIG. 4B illustrates an example of the pre-processing step of manufacturing process parameters (MPP) used in training a probabilistic graphical model to predict the distribution of glycans attached to molecules during a biomolecules manufacturing process. It shows a graph of raw/unprocessed MPP (Lactate) measured during two different batches.

FIG. 4C also illustrates an example of the pre-processing step of manufacturing process parameters (MPP) used in training a probabilistic graphical model to predict the distribution of glycans. It shows a table listing the value of the MPP (Lactate) after different preprocessing steps for the same two batches.

FIGS. 5A-5F show illustrations of the training and validation results, including graphs of measured versus predicted distribution of glycans. The Tables list the various performance evaluation metrics of the disclosed probabilistic graphical model to predict the distribution of glycans attached to biomolecules during a biomolecules manufacturing process, in accordance with various embodiments.

FIG. 6 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Mammalian and other cell lines are frequently used in the production of desired biological molecules. Cell culture operating parameter optimization helps achieve high expression of product with acceptable product quality profiles. These parameters are physical, chemical and biological in nature. Physical parameters include, but are not limited to, temperature, gas flow rate and agitation speed; Chemical parameters include, but are not limited to, dissolved oxygen and carbon dioxide, pH, osmolality, redox potential and metabolite levels, including substrate, amino acid and waste by-products; Biological parameters are used for determining the physiological state of the culture and include, but are not limited to, viable cell concentration (VCC), cell viability, a variety of intracellular and extra-cellular measurements such as NADH, LDH levels, mitochondrial activity, cell cycle analysis, etc. to name a few. Variations in the micro-environment parameters from optimal levels can have a dramatic impact on culture performance, productivity and product quality. A typical stirred tank bioreactor is equipped with temperature, pressure, agitation, pH and dissolved oxygen sensors and/or controls. Cell culture operating strategies and parameters that effect cell culture environmental conditions, include but are not limited to, dissolved oxygen (DO), pH, osmolality, dissolved CO₂, mixing, hydrodynamic shear, etc. The cell culture environment consequently influences process performance which can be measured by parameters such as cell growth, metabolite concentrations, product titer and product quality. These cell culture manufacturing processes are well-described in Li et al., Cell culture processes for monoclonal antibody production, mAbs 2:5, 466-477; September/October 2010; Landes Bioscience; the contents of which are hereby incorporated by reference.

Optimization of biomolecule manufacturing processes means measuring, monitoring and adjusting for example, the cell's health, titer, product quality attributes in the bioreactor. Real-time and near real-time monitoring of cell culture manufacturing processes are part of the process analytical technology (PAT) paradigms for upstream bioprocessing. The responses measured can enable rapid feedback to perturbations that can otherwise lead to batch failures. Historically, real-time monitoring of bioreactor processes monitored parameters such as pH, dissolved oxygen, and temperature, or analytical results such as cell growth and metabolites through manual daily sampling. In order to reduce sample error and to increase throughput, real-time and near real-time instruments have been developed. These and recent advances (including dielectric spectroscopy, NIR, off-gas spectrometry, integrated at-line HPLC, and nanofluidic devices for monitoring cell growth and health, metabolites, titer, etc.) are discussed in Current Opinion in Biotechnology, Volume 71, October 2021, Pages 191-197.

However, the use of machine learning (ML) models in cell culture manufacturing processes is still in its infancy. So far, Raman probes have been utilized in ML models. No ML models have been developed to monitor or to predict cell viability so far.

Various embodiments encompassed herein provide methods and systems that utilize training of a probabilistic graphical model for predicting distribution of glycans attached to the biological molecules during manufacturing in a cell-based system. Glycans can be quality attributes of manufacturing processes, and as such, determining the distribution of glycans attached to the molecules using a trained probabilistic graphical model can have several advantages. For example, the disclosed techniques for measuring and predicting glycan distributions improve upon present methods by allowing one to circumvent costly, time-consuming methods for testing for glycan distributions during the process. They also provide for streamlining analysis processes that otherwise would be subject to disadvantages such as in-process sampling and testing steps fraught with the potential for contamination, for example. In addition, because the techniques allow the determination of glycan distributions much faster (e.g., in real-time) than is possible via present methods (e.g., manual measurements), the manufacturing process can be highly expedited, saving time and resources. However, in some embodiments, in-process sampling and testing steps may also be utilized.

In various embodiments, the glycan distribution on biomolecules in cell culture for the present disclosure is an example of a measure of the output of the desired biological molecules. The disclosure encompasses embodiments for production of any type of biomolecule, including proteins (biopharmaceuticals, cytokines, fusion proteins, growth factors, monoclonal antibodies, complex antibodies, antibody fragments, virus or viral particles, vaccines, etc.); lipids; carbohydrates; and nucleic acids. The cells in the cell culture produce the monoclonal antibody, complex antibody, antibody fragment, virus or viral particles, vaccines, etc.

The present disclosure encompasses methods and systems that utilize training of a probabilistic graphical model for predicting distribution of glycans attached to biomolecules, such as immunotherapeutic molecules including, but not limited to, antibodies that are being manufactured in a cell-based system. In particular embodiments, glycosylation of antibodies represents a key source of heterogeneity that can influence safety and efficacy for the antibody, and the present disclosure encompasses systems and methods trained on glycosylation pattern data from the model(s).

Therapeutic monoclonal antibodies that recognize antigens on the surface of cells do so by binding Fcγ receptors (FcγR) on effector cells (NK cells, monocytes, etc.), thereby eliciting antibody-dependent cell-mediated cytotoxicity (ADCC). The different FcγR may be classified as activating (FcγRI, FcγRIIa and FcγRIII) or inhibitory (FcγRIIb). The heavy chain of antibodies comprises biantennary N-glycan structures linked to Asn297 in the CH2 domain. Each of the CH2 domains on the antibodies often have different glycan chains linked, and these impact binding of the fraction crystallizable (Fc) to the Fc-receptors. In generating antibody proteins in recombinant cellular systems, the proteins often have complex type biantennary oligosaccharides in the Fc portions that often comprise a core fucose and a bisecting N-acetylglucosamine, and variations in terminal galactose and sialic acid may also be present.

The activities of therapeutic antibodies can be impacted by the Fc glycosylation pattern, including patterns and outcome affected by, e.g., galactosylation, core-fucose levels, or lack thereof, etc., Such characteristics can influence ADCC activity, thereby affecting clinical efficacy. This correlation is manipulated in glycoengineering as one way to improve efficacy, such as to produce antibodies lacking core focuses that allow better binding affinity with FcγRIIIa, leading to improved ADCC activity.

The present disclosure encompasses a complex yet efficacious system and related methods in which decision-making for one or more parameters can occur (prior to and/or) in real-time. In various embodiments, the distribution of glycans attached to certain molecules are related to the cell culture system and manufacturing output, and based on the trained probabilistic graphical model one can modify, manipulate, or toggle one or more parameters to better predict or manipulate the glycosylation distribution. In particular embodiments, for at least some parameters this can occur in the absence of testing, thereby maintaining the integrity of the cell-based system and saving cost and labor and risk of contamination. The present system and processes allow for manipulation of choice of parameter, order of parameter inputs, magnitude of parameter content, a combination thereof, etc., as a result of predictive information from the model. Such manipulations occur at the process conditional level instead of the cellular level, as with known cell-based manufacturing systems, and allow for prompt modification(s) dependent upon one or more outputs, for example.

In various embodiments, the methods and systems concern predicting the glycosylation distribution for monoclonal antibodies for detection, diagnostic, and/or therapeutic purposes. In specific embodiments, the methods predict the distribution of glycans attached to molecules such as monoclonal antibodies based on a process of manufacturing antibodies in a cell culture in a bioreactor. The cell culture environment allows analysis of antibody-producing cells in the presence of one or more manipulable process parameters, and values for such parameters are input into a trained probabilistic graphical model that provides an informative output(s) that facilitates prediction of glycan distribution of the antibodies. In specific embodiments, the encompassed methods train a probabilistic graphical model based on the distribution of glycans attached to the antibody that may be applied to subsequent antibodies.

### II. Definitions

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “about” refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, ±5%, or 1% as understood by a person of skill in the art.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more graphical models, one or more machine learning algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

As used herein, “probabilistic graphical model” may refer to representations of probability distributions over several variables that interact with each other. Probabilistic graphical models may include Bayesian networks, which use directed graphs, or Markov networks, which use undirected graphs.

The term “antibody” as used herein refers to any immunologic binding agent such as IgG, IgM, IgA, IgD and IgE and also refers to any antibody-like molecule that has an antigen binding region, and includes antibody fragments such as Fab′, Fab, F(ab′)2, single domain antibodies (DABs), Fv, scFv (single chain Fv), and the like. The antibody may be monoclonal or humanized, in specific embodiments.

The term “bioreactor” as used herein refers to an apparatus suitable for housing and growing a cell culture, including on a manufacturing scale, in some embodiments.

The term “cell culture” as used herein refers to growth of cells in an artificial environment under suitable conditions.

The term “molecule” as used herein refers to substances that are produced by cells and may include carbohydrates, lipids, nucleic acids, antibodies (e.g., monoclonal), and proteins. In embodiments, molecule or biomolecule refers to a monoclonal antibody, a complex antibody, an antibody fragment, a virus or a viral particle, lipids; carbohydrates; nucleic acids, mixtures thereof, etc.

The term “glycan” as used herein refers to carbohydrate structures that are attached to biomolecules (e.g., monoclonal antibodies). These carbohydrate structures may be attached to the same location on the molecules.

The term “glycan distribution” as used herein refers to the relative proportions of glycans attached to the same location on a biomolecule.

### III. Prediction of Distribution of Glycans Attached to Molecules Produced Via a Biomolecule Manufacturing Process

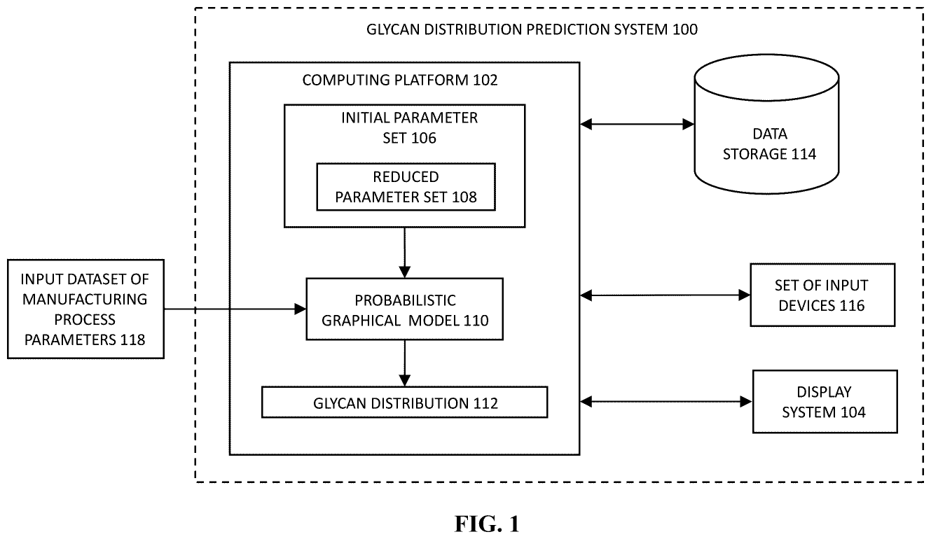

FIG. 1 is a block diagram of a glycan distribution prediction system 100 in accordance with various embodiments. Glycan distribution prediction system 100 uses a probabilistic graphical model 110 to predict distribution of glycans attached to biomolecules that are produced via a biomolecules manufacturing process in a cell culture in a bioreactor. In some instances, the manufacturing process can be a biomolecule manufacturing batch process or continuous process (e.g., perfusion process). In specific embodiments, the process is noninvasive. In various embodiments, the process allows for continuous or periodic monitoring and adjustment to maintain optimum conditions within the bioreactor. In specific embodiments, the process allows for maintaining optimum nutrient and waste levels in the culture, including within predefined acceptable ranges. In particular aspects there is monitoring of any stage of the process in real time. In particular embodiments, the system can use an open loop or closed loop control for monitoring one or more manufacturing process parameters and then automatically change or vary one or more components, such as, e.g., flow of a parameter component in or out of the bioreactor. In some embodiments, the change may include flow in of a first parameter component and flow out of a second or subsequent parameter component that may or may not occur substantially at the same time. Any manufacturing process parameter may comprise, in certain embodiments, any fluid, compound, molecule, or substance that can increase the mass of the manufactured molecule in the cell culture.

Examples of the molecules include but are not limited to therapeutic antibodies (e.g., monoclonal antibodies (mAbs)). Glycan distribution prediction system 100 may include computing platform 102, data storage 114, set of input devices 116, and display system 104.

Computing platform 102 may take various forms. In various embodiments, computing platform 102 includes a single computer (or computer system) or multiple computers in communication with each other. In other examples, computing platform 102 takes the form of a cloud computing platform. In various embodiments, computing platform 102 may be communicatively coupled with data storage 114, set of input devices 116, display system 104, or a combination thereof. In various embodiments, data storage 114, set of input devices 116, display system 104, or a combination thereof may be considered part of or otherwise integrated with computing platform 102. Thus, in some examples, computing platform 102, data storage 114, set of input devices 116, and display system 104 may be separate components in communication with each other, but in other examples, some combination of these components may be integrated together.

In various embodiments, the initial parameter set 106 may include manufacturing process parameters that are related to the process of manufacturing the biomolecules in the cell culture in the bioreactor. In some instances, these manufacturing process parameters can be measured during the manufacturing process and/or obtained from a database (e.g., from data storage 114) coupled to the computing platform 102. For example, molecules such as but not limited to mAbs can be produced in a bioreactor, and parameters related to the manufacturing process of such molecules in the reactor may be measured or otherwise obtained (e.g., and stored in data storage 114). In some instances, the measurements can be online (e.g., measured using sensors or controllers coupled to the bioreactor) and/or offline (e.g., measured manually, using samples, for instance).

Non-limiting examples of said manufacturing process parameters include amount of carbon dioxide sparged into the cell culture, amount of air sparged into the cell culture, amount of oxygen sparged into the cell culture, pH of the cell culture, concentration of dissolved oxygen (dO₂) in the bioreactor, total base added into the cell culture during the biomolecules manufacturing process (“base total”), agitation control output, back pressure, total volume of the cell culture, temperature of the cell culture, the temperature of the bioreactor/vessel shell (“jacket temperature”), temperature output of a bioreactor/vessel shell controller, culture duration (e.g., time elapsed since commencement of cell culture process), glucose concentration, lactate concentration, sodium concentration, ammonium concentration, osmolality, packed cell volume (PCV), carbon dioxide concentration, oxygen concentration, conductivity of the cell culture, percent cell viability, viable cell density (VCD), the packed cell volume (PCV), concentration of immunoglobulin G (IgG), specific production of IgG (qIgG), cell growth rate, glucose uptake rate (GUR), and/or the like. In various embodiments, one, some or all of these manufacturing process parameters may be measured repeatedly during the culture duration, and as such rates of the manufacturing process parameters may be measured or computed. For example, by measuring the VCD multiple times over the culture duration, VCD/time may be obtained.

As used herein, the CO₂ sparge refers to the introduction of CO₂ bubbled through the cell culture. In specific embodiments, CO₂ is sparged in as a pH control mechanism to maintain pH at a set point. Base may be added to the cell culture to increase pH, while CO₂ and/or acid may be added to decrease pH. Since even a small deviation of 0.1 units from the optimal pH value can significantly impact culture growth and metabolism, in particular glucose consumption and lactate production, pH is an important variable to measure and control. Cell culture media usually contains sodium bicarbonate as buffering agent, and pH is usually tightly controlled with a combination of CO₂ sparging to reduce pH and base addition to increase it. High pH (>7.0) is usually preferred for initial cell growth phase, which is usually accompanied by lactate accumulation. When lactate accumulation exceeds the buffering capacity of the culture medium, pH drifts downward, which could trigger base addition leading to increased osmolality of the culture medium. This could be risky in cell lines that synthesize excessive amounts of lactate since high pH, high lactate and high osmolality cascade often causes delayed cell growth and accelerated cell death. When cell growth has ceased, lactate is either produced at a much lower rate or consumed. The concomitant upward drift in pH is counteracted by CO₂ sparging. Thus, the pH set-point and control strategy, e.g., dead band, are intimately linked to dissolved CO₂ levels, base consumption for pH control and therefore, osmolality (from Li et al., mAbs 2:5, 466-477; September/October 2010; Landes Bioscience; the contents of which are hereby incorporated by reference). As used herein, the O₂ sparge refers to the introduction of O₂ bubbled through the cell culture to support cell respiration and growth. Dissolved oxygen is typically controlled at a specific set point, usually between 20-50% of air saturation in order to prevent dissolved oxygen limitation, which might lead to excessive lactate synthesis, and excessively high dissolved oxygen concentrations that could lead to cytotoxicity.

As used herein, the air sparge refers to the introduction of air bubbled through the cell culture.

In various embodiments, the pH of the cell culture in the cell culture in the bioreactor may be in a range of from about 6.5 to about 7.5 (also refer to CO₂ sparging above and pH control). In some embodiments, the pH of the cell culture may be in a range from about 6.5 to about 7.4, about 6.5 to about 7.3, about 6.5 to about 7.2, about 6.5 to about 7.1, about 6.5 to about 7.0, about 6.5 to about 6.9, about 6.5 to about 6.8, about 6.5 to about 6.7, about 6.5 to about 6.6, about 6.6 to about 7.5, about 6.6 to about 7.4, about 6.6 to about 7.3, about 6.6 to about 7.2, about 6.6 to about 7.1, about 6.6 to about 7.0, about 6.6 to about 6.9, about 6.6 to about 6.8, about 6.6 to about 6.7, about 6.7 to about 7.5, about 6.7 to about 7.4, about 6.7 to about 7.3, about 6.7 to about 7.2, about 6.7 to about 7.1, about 6.7 to about 7.0, about 6.7 to about 6.9, about 6.7 to about 6.8, about 6.8 to about 7.5, about 6.8 to about 7.4, about 6.8 to about 7.3, about 6.8 to about 7.2, about 6.8 to about 7.1, about 6.8 to about 7.0, about 6.8 to about 6.9, about 6.9 to about 7.5, about 6.9 to about 7.4, about 6.9 to about 7.3, about 6.9 to about 7.2, about 6.9 to about 7.1, about 6.9 to about 7.0, about 7.0 to about 7.5, about 7.0 to about 7.4, about 7.0 to about 7.3, about 7.0 to about 7.2, about 7.0 to about 7.1, about 7.1 to about 7.5, about 7.1 to about 7.4, about 7.1 to about 7.3, about 7.1 to about 7.2, about 7.2 to about 7.5, about 7.2 to about 7.4, about 7.2 to about 7.3, about 7.3 to about 7.5, about 7.3 to about 7.4, or about 7.4 to about 7.5. In specific embodiments, the pH of the cell culture may be at least, or no more than, about 6.5, about 6.6, about 6.7, about 6.8, about 6.9, about 7.0, about 7.1, about 7.2, about 7.3, about 7.4, or about 7.5. In some instances, the term “pH primary” may be used to refer to the pH of the cell culture that is measured by a pH sensor onboard the bioreactor (e.g., measuring “online” in real-time), while the term “pH output” may be used to refer to pH values measured by a pH controller or sensor (e.g., and may be used to determine whether base or CO₂ should be adjusted to increase or decrease the pH of the cell culture).

In certain embodiments, the amount or concentration of dissolved oxygen (dO₂) in the cell culture may be in a range of from about 0% to about 100%, from about 20% to about 80%, from about 40% to about 60%, including values and subranges therebetween. Dissolved oxygen is typically controlled at a specific set point, usually between 20-50% of air saturation. The dO₂ may be in a range of 0% to about 80%, 0% to about 75%, 0% to about 60%, 0% to about 50%, 0% to about 40%, 0% to about 20%, 0% to about 10%, !% to about 5%, about 5% to about 100%, about 5% to about 80%, about 5% to about 75%, about 5% to about 60%, about 5% to about 50%, about 5% to about 40%, about 5% to about 20%, about 5% to about 10%, about 10% to about 100%, about 10% to about 80%, about 10% to about 75%, about 10% to about 60%, about 10% to about 50%, about 10% to about 40%, about 10% to about 20%, about 20% to about 100%, about 20% to about 80%, about 20% to about 75%, about 20% to about 60%, about 20% to about 50%, about 20% to about 40%, about 40% to about 100%, about 40% to about 80%, about 40% to about 75%, about 40% to about 60%, about 40% to about 50%, about 50% to about 100%, about 50% to about 80%, about 50% to about 75%, about 50% to about 60%, about 60% to about 100%, about 60% to about 80%, about 60% to about 75%, about 75% to about 100%, about 75% to about 80%, or about 80% to about 100%. In some embodiments, the dO₂ may be at least, or no more than, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, the temperature of the cell culture may be in a range of from about 30° C. to about 37° C. In some embodiments, the temperature may be in the range of about 30° C. to about 36° C., about 30° C. to about 35° C., about 30° C. to about 34° C., about 30° C. to about 33° C., about 30° C. to about 32° C., about 30° C. to about 31° C., about 31° C. to about 37° C., about 31° C. to about 36° C., about 31° C. to about 35° C., about 31° C. to about 34° C., about 31° C. to about 33° C., about 31° C. to about 32° C., about 32° C. to about 37° C., about 32° C. to about 36° C., about 32° C. to about 35° C., about 32° C. to about 34° C., about 32° C. to about 33° C., about 33° C. to about 37° C., about 33° C. to about 36° C., about 33° C. to about 35° C., about 33° C. to about 34° C., about 34° C. to about 37° C., about 34° C. to about 36° C., about 34° C. to about 35° C., about 35° C. to about 37° C., about 35° C. to about 36° C., or about 36° C. to about 37° C. In some embodiments, the temperature may be at least, or no more than, about 30° C., about 31° C., about 32° C., about 33° C., about 34° C., about 35° C., about 36° C., or about 37° C.

In certain embodiments, the culture duration may be from about 3 days to about 30 days, from about 5 days to about 25 days, from about 5 days to about 20 days, from about 6 days to about 16 days, from about 8 days to about 12 days, from about 10 days to about 12 days, including values and subranges therebetween. In some embodiments, the culture duration may be from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days. In some embodiments, the culture duration may be at least, or no more than, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days.

In various embodiments, the concentration of glucose in the media of the culture may be in a range from about 0 g/L to about 50 g/L, from about 0 g/L to about 40 g/L, from about 0 g/L to about 30 g/L, from about 0 g/L to about 20 g/L, from about 0 g/L to about 10 g/L, from about 10 g/L to about 50 g/L, from about 10 g/L to about 40 g/L, from about 10 g/L to about 30 g/L, from about 10 g/L to about 20 g/L, from about 20 g/L to about 50 g/L, from about 20 g/L to about 40 g/L, from about 20 g/L to about 30 g/L, from about 30 g/L to about 50 g/L, from about 30 g/L to about 40 g/L, or from about 40 g/L to about 50 g/L, including values and subranges therebetween. In various embodiments, the amount of glucose in the media of the culture may be at least, or no more than, about 0 g/L, about 10 g/L, about 20 g/L, about 30 g/L, about 40 g/L, or about 50 g/L. In various embodiments, the concentration of lactate produced by cells in the media may be an indirect measure for cell growth of the culture and may be in a range from about 0 g/L to about 50 g/L, from about 0 g/L to about 40 g/L, from about 0 g/L to about 30 g/L, from about 0 g/L to about 20 g/L, from about 0 g/L to about 10 g/L, from about 10 g/L to about 50 g/L, from about 10 g/L to about 40 g/L, from about 10 g/L to about 30 g/L, from about 10 g/L to about 20 g/L, from about 20 g/L to about 50 g/L, from about 20 g/L to about 40 g/L, from about 20 g/L to about 30 g/L, from about 30 g/L to about 50 g/L, from about 30 g/L to about 40 g/L, or from about 40 g/L to about 50 g/L, including values and subranges therebetween. In various embodiments, the amount of lactate in the media of the culture may be at least, or no more than, about 0 g/L, about 10 g/L, about 20 g/L, about 30 g/L, about 40 g/L, or about 50 g/L. In some embodiments, lactate is a waste product in cell culture, and so the amount of lactate is monitored to ensure that the level of lactate in the culture is no more than a particular concentration, such as no more than about 0 g/L, about 5 g/L, about 10 g/L, about 15 g/L, about 20 g/L, about 25 g/L, about 30 g/L, about 35 g/L, about 40 g/L, about 45 g/L, or about 50 g/L.

In certain embodiments, the concentration of sodium in the media may be in a range of from about 0 to about 250 mM, about 0 to about 200 mM, about 0 to about 150 mM, about 0 to about 100 mM, about 0 to about 50 mM, about 0 to about 25 mM, about 25 mM to about 250 mM, about 25 mM to about 200 mM, about 25 mM to about 150 mM, about 25 mM to about 100 mM, about 25 mM to about 50 mM, about 50 mM to about 250 mM, about 50 mM to about 200 mM, about 50 mM to about 150 mM, about 50 mM to about 100 mM, about 100 mM to about 250 mM, about 100 mM to about 200 mM, or about 200 mM to about 250 mM, including values and subranges therebetween. In some embodiments, the amount of sodium in the media may be at least, or no more than, 5 mM, 10 mM, 15 mM, 20 mM, 25 mM, 50 mM, 75 mM, 100 mM, 125 mM, 150 mM, 175 mM, 200 mM, 225 mM, or 250 mM.

In certain embodiments, the concentration of ammonium in the media may be in a range of from about 0 to about 50 mM, including values and subranges therebetween. In certain embodiments, the amount of ammonium in the media may be in a range of from about 0 to about 40 mM, about 0 to about 30 mM, about 0 to about 20 mM, about 0 to about 10 mM, about 10 mM to about 50 mM, about 10 mM to about 40 mM, about 10 mM to about 30 mM, about 10 mM to about 20 mM, about 20 mM to about 50 mM, about 20 mM to about 40 mM, about 20 mM to about 30 mM, about 30 mM to about 50 mM, about 30 mM to about 40 mM, or about 40 mM to about 50 mM. In some embodiments, the amount of ammonium in the media may be at least, or no more than, 0, 5 mM, 10 mM, 15 mM, 20 mM, 25 mM, 30 mM, 35 mM, 40 mM, 45 mM, or 50 mM.

In various embodiments, the osmolality of the culture may be in a range of from about 0 to about 700 mOsm, including values and subranges therebetween. In some embodiments, the osmolality of the culture may be in a range of from about 0 mOsm to about 700 mOsm, from about 0 mOsm to about 600 mOsm, from about 0 mOsm to about 500 mOsm, from about 0 mOsm to about 400 mOsm, from about 0 mOsm to about 300 mOsm, from about 0 mOsm to about 200 mOsm, from about 0 mOsm to about 100 mOsm, from about 0 mOsm to about 50 mOsm, from about 50 mOsm to about 700 mOsm, from about 50 mOsm to about 600 mOsm, from about 50 mOsm to about 500 mOsm, from about 50 mOsm to about 400 mOsm, from about 50 mOsm to about 300 mOsm, from about 50 mOsm to about 200 mOsm, from about 50 mOsm to about 100 mOsm, from about 100 mOsm to about 700 mOsm, from about 100 mOsm to about 600 mOsm, from about 100 mOsm to about 500 mOsm, from about 100 mOsm to about 400 mOsm, from about 100 mOsm to about 300 mOsm, from about 100 mOsm to about 200 mOsm, from about 200 mOsm to about 700 mOsm, from about 200 mOsm to about 600 mOsm, from about 200 mOsm to about 500 mOsm, from about 200 mOsm to about 400 mOsm, from about 200 mOsm to about 300 mOsm, from about 300 mOsm to about 700 mOsm, from about 300 mOsm to about 700 mOsm, from about 300 mOsm to about 600 mOsm, from about 300 mOsm to about 500 mOsm, from about 300 mOsm to about 400 mOsm, from about 400 mOsm to about 700 mOsm, from about 400 mOsm to about 600 mOsm, from about 400 mOsm to about 500 mOsm, from about 500 mOsm to about 700 mOsm, or from about 600 mOsm to about 700 mOsm, In specific embodiments, the osmolality of the culture may be at least, or no more than, 0 mOsm, 25 mOsm, 50 mOsm, 75 mOsm, 100 mOsm, 150 mOsm, 200 mOsm, 250 mOsm, 250 mOsm, 300 mOsm, 350 mOsm, 400 mOsm, 450 mOsm, 500 mOsm, 550 mOsm, 600 mOsm, 650 mOsm, or 700 mOsm.

In various embodiments, the carbon dioxide of the culture may be in a range of from about 0 to about 250 mmHg, including values and subranges therebetween. In some embodiments, the carbon dioxide of the culture may be in a range of from about 0 to about 250 mmHg, about 0 to about 200 mmHg, about 0 to about 150 mmHg, about 0 to about 100 mmHg, about 0 to about 50 mmHg, about 50 mmHg to about 250 mmHg, about 50 mmHg to about 200 mmHg, about 50 mmHg to about 150 mmHg, about 50 mmHg to about 100 mmHg, about 100 mmHg to about 250 mmHg, about 100 mmHg to about 200 mmHg, about 100 mmHg to about 150 mmHg, about 150 mmHg to about 250 mmHg, about 150 mmHg to about 200 mmHg, or about 150 mmHg to about 250 mmHg. In some embodiments, the carbon dioxide of the culture may be at least, or no more than 1, 5 mmHg, 10 mmHg, 15 mmHg, 20 mmHg, 25 mmHg, 30 mmHg, 35 mmHg, 40 mmHg, 45 mmHg, 50 mmHg, 75 mmHg, 100 mmHg, 125 mmHg, 150 mmHg, 175 mmHg, 200 mmHg, 225 mmHg, or 250 mmHg.

In various embodiments, the oxygen (02) of the culture may be in a range of from about 0 to about 250 mmHg, including values and subranges therebetween. In some embodiments, the 02 of the culture may be in a range of from about 0 to about 250 mmHg, about 0 to about 200 mmHg, about 0 to about 150 mmHg, about 0 to about 100 mmHg, about 0 to about 50 mmHg, about 50 mmHg to about 250 mmHg, about 50 mmHg to about 200 mmHg, about 50 mmHg to about 150 mmHg, about 50 mmHg to about 100 mmHg, about 100 mmHg to about 250 mmHg, about 100 mmHg to about 200 mmHg, about 100 mmHg to about 150 mmHg, about 150 mmHg to about 250 mmHg, about 150 mmHg to about 200 mmHg, or about 150 mmHg to about 250 mmHg. In some embodiments, the 02 of the culture may be at least, or no more than 1, 5 mmHg, 10 mmHg, 15 mmHg, 20 mmHg, 25 mmHg, 30 mmHg, 35 mmHg, 40 mmHg, 45 mmHg, 50 mmHg, 75 mmHg, 100 mmHg, 125 mmHg, 150 mmHg, 175 mmHg, 200 mmHg, 225 mmHg, or 250 mmHg.

As used herein, “viable cell density (VCD)” refers to viable cell density (#×10⁶ of viable cells/mL of culture), and in various embodiments, the range of VCD is from about 0 to about 10⁷ viable cells/mL of culture, including values and subranges therebetween. In some embodiments, the range of VCD is at least 10², 10³, 10⁴, 10⁵, 10⁶, or 10⁷ or more viable cells/mL.

As used herein, “PCV” refers to packed cell volume (volume of cells/total volume)*100, and in various embodiments, is in the range of from about 0% to about 25%, from about 0% to about 20%, from about 0% to about 15%, from about 0% to about 10%, from about 10% to about 25%, from about 10% to about 20%, from about 10% to about 15%, 15% to about 25%, 15% to about 20%, or 20% to about 25%, including values and subranges therebetween. In some embodiments, the PCV is at least, or no more than, about 1%, about 5%, about 10%, about 15%, about 20%, or about 25%.

In some embodiments, the total volume of the cell culture comprises a volume suitable to allow proliferation of cells to a desired concentration in the culture. Examples of volumes include at least, or no more than, 100 mL to 500 mL, 100 mL to 1 L, 100 mL to 2 L, 100 mL to 5 L, 100 mL to 10 L, 100 mL to 20 L, 1 L to 3 L, 1 L to 5 L, 1 L to 10 L, or 1 L to 20 L. In some cases, the volume is volume of 20 L or less, 10 L or less, 5 L or less, 4 L or less, 3 L or 15 less, 2 L or less, 1 L or less, or 0.1 L or less. In some embodiments, the volume of the cell culture is at least, or no more than, 500 L, at least 1000 L, at least 2000 L, at least 3000 L, at least 4000 L, at least 5000 L, at least 7500 L, at least 10000 L, at least 12500 L, at least 15000 L, at least 20000 L, at least 100000 L, or more. In some embodiments, the manufacturing-scale bioreactor culture has a working volume of 2000 L or 15000 L. In some embodiments, a manufacturing-scale bioreactor culture has a working volume in a range of 500 L to 1000 L, 500 L to 2500 L, 500 L to 5000 L, 500 L to 10000 L, 500 L to 15000 L, 500 L to 20000 L, 500 L to 100000 L, 2000 L to 5000 L, 2000 L to 10000 L, 2000 L to 15000 L, 2000 L to 20000 L, 2000 L to 100000 L, 15000 L to 20000 L, 15000 L to 100000 L, 20000 L to 50000 L, 20000 L to 100000 L, or 50000 L to 100000 L.

As used herein, the terms “percent viability” or “cell viability” can refer to (# of live cells/# of dead cells)*100. The cells may be any kind of cell and may be tailored to suitability for production of the desired molecule. The cells may be eukaryotic or prokaryotic. The cells may be single-celled organisms, animal cells, or plant cells, as examples.

In various embodiments, as noted above, the initial parameter set 106 may be obtained from online and/or offline measurements associated with the process of manufacturing biomolecules in the bioreactor. That is, the manufacturing process parameters of the initial parameter set 106 may be obtained via online and/or offline measurements. Online measurements can be measurements performed by sensors and/or controllers with outputs that are operationally connected to the bioreactor. Online measurements can be performed or collected very often, for example, continuously or nearly continuously (e.g., ever second or few seconds). Offline measurements can be measurements that are performed using standalone instruments on samples that are extracted from the bioreactor (e.g., manually). Offline measurements can be performed or collected regularly but not often (e.g., every day, every two days, etc.). In some instances where the manufacturing process is a batch process, the measurements can be performed per batch. For example, the measurement can be for amount or distribution of glycans, and the glycan amount or distribution may be measured per batch of molecules manufactured by the process. Other examples of measurements include charge variants, cell culture-related information (e.g., trace metals, media components, additions), etc., which may be measured per batch as well.

Online measurements of may be performed by one or more probes that are operationally coupled to the bioreactor, or cell culture therein, and configured to measure manufacturing process parameters associated with the bioreactor and/or the cell culture. For example, a scale may be operationally coupled to the bioreactor and may be configured to measure the weight or volume of the cell culture. As another example, a temperature probe, a dissolved oxygen probe, and/or a pH probe may be operationally coupled to the bioreactor and/or the cell culture to measure the temperature of the cell culture, the amount or concentration of dissolved oxygen in the cell culture, or the pH of the cell culture, respectively. In some instances, the temperature probe may also be configured to measure the temperatures of other components or process features of the manufacturing process (e.g., in addition to or besides the temperature of the cell culture). For example, the temperature probe may be configured to measure the temperature of the bioreactor/vessel shell (“jacket temperature”).

In some instances, the one or more online measurements may be obtained from controllers configured to control the one or more manufacturing process parameters. For example, a controller that is operationally coupled to the bioreactor and configured to control a manufacturing process parameter may have an output associated with the manufacturing process parameter. In such cases, the manufacturing process parameter may be measured by reading the output of the controller.

In some embodiments, the controller can be in communication and control thermocirculators, load cells, control pumps, and receive information from various sensors and probes. For instance, the controller may control and/or monitor the pH, the oxygen tension, dissolved carbon dioxide, the temperature, the agitation conditions, the alkali condition, the pressure, foam levels, and the like. For example, based on pH readings from a pH probe, the controller may be configured to regulate pH levels by adding requisite amounts of acid or alkali. The controller may also use a carbon dioxide gas supply to decrease pH. Similarly, the controller can receive temperature information and control fluids being feed to a water jacket surrounding the bioreactor for increasing or decreasing temperature.

In various embodiments, for example, the bioreactor may be operationally coupled to an air flow controller configured to control the flow of air, carbon dioxide, oxygen, a combination thereof, etc., sparged into the cell culture, and the output of the controller may be read to measure or determine the amount of air, carbon dioxide, oxygen, etc., respectively, that is sparged into the cell culture. As another example, a temperature controller may be configured to control the temperature of the bioreactor vessel, and the output of the temperature controller may be read to measure the jacket temperature and/or the temperature of the cell culture. In some instances, a pH controller may be configured to control the pH of the cell culture, and the reading of this pH controller (referred herein as “pH output”) may be used as a measure of the pH of the cell culture. In some cases, the pH output measurement may be used to regulate the pH of the cell culture. For example, based on the pH output measurement, a base may be added, or CO₂ sparged (and/or acid added), into the cell culture, to increase or decrease the pH of the cell culture, respectively.

In certain cases, the one or more offline parameters, such as glucose concentration, lactate concentration, sodium concentration, ammonium concentration, a combination thereof, etc., may be obtained by respective commercial kits (e.g., Cedex analyzer from Roche). Although buffers are included in the bioreactor to control acidity of the culture media, production of metabolic and other acids by the cells may upset the equilibrium of the culture and require adjustment of the one or more parameters (e.g., pH) by standard methods. In some embodiments, the base total may be measured based on the rate that base is added into the cell culture and the duration of the addition (e.g., the product of the rate and the duration gives the base total). In certain aspects, the osmolality may be measured using an offline freezing point osmometer. In one example, a controller is operationally coupled to the bioreactor and configured to control the intensity of agitation as agitation control output. In some cases, a controller that regulates back pressure is utilized and may comprise a normally-closed valve configured to provide an obstruction or a pressure hold to flow, thereby regulating the upstream (back) pressure. “Back” in this context means against the natural flow of fluid and refers to upstream pressure that is held, or maintained, in a variety of production vessels to provide the right conditions for separation and processing. As one example, one or more gas sensors may be operationally coupled to the bioreactor and configured to sense and regulate the amount of gas in the bioreactor, including for carbon dioxide and oxygen. The PCV in the bioreactor may be measured using commercially available tubes and equipment for the determination of biomass (such as a micro capillary centrifuge). Or, cell density can also be measured using cell counter sensor or an optical density sensor.

In various embodiments, the initial parameter set 106 may include manufacturing process parameters that are part of a model training records or dataset that can be used to train the probabilistic graphical model 110 to predict an output of the biomolecules manufacturing process. An example of the output can be the glycan distribution 112 of glycans attached to a location or locations on the molecules that are produced by the molecules manufacturing process. For instance, as discussed above, the initial parameter set 106 may include a variety of manufacturing process parameters that are measured (e.g., via sensors, probes, controllers, combinations thereof, etc.) or otherwise obtained during the manufacturing of molecules in a cell culture in a bioreactor. In such cases, the glycan distribution of the molecules produced via the manufacturing process may also be measured or otherwise obtained (e.g., a manually sampled cell culture may be analyzed to determine the glycan distribution). The training dataset can then include the manufacturing process parameters paired with the measured glycan distribution. The training of the probabilistic graphical model then comprises inputting the training dataset (e.g., which includes the manufacturing process parameters of the initial parameter set 106 and the measured glycan distribution) into the probabilistic graphical model 110 to train the probabilistic graphical model to predict the measured glycan distribution.

In some instances, it may be desirable to remove one or more of the manufacturing process parameters in the training dataset, such as to reduce the size of the training dataset. For example, the one or more of the manufacturing process parameters to be removed may have low importance for or impact on the training of the probabilistic graphical model 110, and/or may not have unique contribution to the training of the probabilistic graphical model 110. For instance, two manufacturing process parameters may be highly correlated and as such may have at least substantially similar contribution (e.g., provide substantially similar information) towards the training of the probabilistic graphical model 110. In such cases, it may be desirable to remove from the training dataset those redundant and/or low-importance manufacturing process parameters, such as to save computing resources and time as well as reduce the complexity of training of the model.

In various embodiments, the reduced parameter set 108 may be obtained from the initial parameter set 106 by pre-processing the latter using model feature selection techniques that are formulated to remove one or more of the manufacturing process parameters (e.g., those that are redundant, those that have low impact on the training of the probabilistic graphical model 110, a combination thereof, and/or the like). In some instances, the term “features” refers to the manufacturing process parameters in the training dataset, and examples of feature selection techniques include filter methods, wrapper methods, principal component analysis (PCA), or combination thereof. A reduced training dataset may then be generated based on the reduced parameter set 108 and the glycan distribution associated with the manufacturing process parameters of the reduced parameter set 108.

Filter methods include statistical techniques that measure the importance of features in a dataset for training the probabilistic graphical model. As such, the application of filter methods to the initial parameter set 106 includes the application of the statistical techniques to measure the importance of the manufacturing process parameters in the initial parameter set 106 to the training the probabilistic graphical model. In such cases, the manufacturing process parameters identified as being of low importance based on the statistical techniques may be removed from the initial parameter set 106 to generate the reduced parameter set 108. The reduced training dataset may then include the reduced parameter set 108 and the glycan distribution associated with the manufacturing process parameters of the reduced parameter set 108.

The statistical techniques in the filter methods include the computations of information gain/mutual information, Fisher score, correlation coefficient, variance threshold, and/or the like. Information gain or mutual information refers to the amount of information that is obtained by a model from a manufacturing process parameter for use by the model in predicting the target variable, i.e., the glycan distribution. Information gain or mutual information measures the dependency of the manufacturing process parameter over the glycan distribution. An information gain that is zero indicates that the manufacturing process parameter and the glycan distribution are independent variables (i.e., no information about the glycan distribution can be obtained from the manufacturing process parameter). In some instances, manufacturing process parameters with information gain less than a threshold amount may be removed from the initial parameter set 106 to generate the reduced parameter set 108 (e.g., and consequently the reduced training dataset for training the probabilistic graphical model 110).

In some instances, Fisher scores of the manufacturing process parameters in the initial parameter set 106 can be computed to evaluate the individual importance of the manufacturing process parameters to the training of the probabilistic graphical model 110. A larger or smaller Fisher score associated with a manufacturing process parameter indicates that the manufacturing process parameter has higher or lower importance, and in such cases, manufacturing process parameters of the initial parameter set 106 with Fisher score no greater than a threshold Fisher score may be removed from the initial parameter set 106 to generate the reduced parameter set 108 (e.g., and consequently the reduced training dataset for training the probabilistic graphical model 110).

Filter methods can utilize correlation coefficients to measure linear relationships or correlations between any two manufacturing process parameters in the initial parameter set 106. Such multicollinearity analyses allow the identification of correlations between manufacturing process parameters, facilitating the reduction of the initial parameter set 106 to the reduced parameter set 108 by the removal of redundant manufacturing process parameters. Larger or smaller correlation coefficients between pairs of manufacturing process parameters indicate higher or lower, respectively, correlations between the pairs of manufacturing process parameters. Because highly correlated manufacturing process parameters contribute or provide largely similar information towards the training of the probabilistic graphical model 110, larger correlation coefficients may be understood to indicate parameter redundancies in the initial parameter set 106. In some instances, when a pair of manufacturing process parameters have associated therewith a correlation coefficient exceeding a threshold correlation coefficient, one of the pair may be removed from the initial parameter set 106 to generate the reduced parameter set 108 (e.g., and consequently the reduced training dataset for training the probabilistic graphical model 110).

In various embodiments, correlations between the manufacturing process parameters and the glycan distribution predicted by the probabilistic graphical model 110 may also be used to generate the reduced parameter set 108 from the initial parameter set 106. For example, a principal component analysis may be performed to identify and remove from the initial parameter set 106 manufacturing process parameters that have very low correlation with the glycan distribution. In some instances, some manufacturing process parameters may have low correlation with most or all of the glycans under consideration (e.g., the below-identified eight glycans). That is, these manufacturing process parameters may have low correlation with the glycan distribution, and as such may be removed from the initial parameter set 106 to form the reduced parameter set 108. Manufacturing process parameters that may be retained in the reduced parameter set 108 include those that have high correlation with the relative proportions of most or all of the glycans (e.g., those that have high correlation with the glycan distribution).

In some instances, filter methods include computations configured to evaluate the variability of the manufacturing process parameters in the initial parameter set 106. Because manufacturing process parameters that have low variance are expected to have low contribution towards the training of the probabilistic graphical model 110 to predict the glycan distribution 112, manufacturing process parameters with variance less than a variance threshold may be removed from the initial parameter set 106 to generate the reduced parameter set 108 (e.g., and consequently the reduced training dataset for training the probabilistic graphical model 110). In some instances, the variance of a manufacturing process parameter X may be computed using the expression Var [X]=E[(X−E[X])²], where E(X) is the expected value of the manufacturing process parameter X.

In various embodiments, wrapper methods of selecting manufacturing process parameters from the initial parameter set 106 to form the reduced parameter set 108 utilize algorithms that evaluate the performance of the probabilistic graphical model 110 for different subsets of the initial parameter set 106. In some instances, different subsets of the manufacturing process parameters of the initial parameter set 106 may be formed and these subsets may be provided to the probabilistic graphical model 110 as inputs for predicting the glycan distribution 112. The performance of the subsets may be evaluated by comparing the predicted glycan distribution with the measured glycan distribution that is associated with the initial parameter set 106. When the predicted glycan distribution substantially matches the measured glycan distribution, the subset of manufacturing process parameters that resulted in the predicted glycan distribution may be viewed as an optimal set of manufacturing process parameters to use in training the probabilistic graphical model 110. In such cases, the reduced parameter set 108 may be the same as or may be formed based on that subset of manufacturing process parameters.

In various embodiments, PCA may be performed to reduce the dimensionality of the initial parameter set 106. For example, the manufacturing process parameters of the initial parameter set 106 may be linearly combined to construct principal components that are uncorrelated and ordered based on their significance to the prediction of the glycan distribution by the probabilistic graphical model 110. The ordering can be according to the variance in the initial parameter set 106. The number of principal components in the analysis may be chosen to reduce the dimensionality of the training dataset while but avoid or minimize the loss of information. The training dataset in the original basis (e.g., the initial parameter set 106) may then be transformed to the basis of the principal components, referred to as PCA scores (e.g., the reduced parameter set 108). The transformed or reduced training dataset 108 may then be provided to the probabilistic graphical model 110 to train the probabilistic graphical model 110 to predict glycan distributions.

In various embodiments, the initial parameter set 106 can be further reduced to generate the reduced parameter set 108 by combining some of the manufacturing process parameters into new variables that may be scale-invariant (e.g., applicable to various scales of molecules manufacturing). For example, at least some of the manufacturing process parameters of the initial parameter set 106 may be combined with the total volume of the cell culture, the VCD, etc., to generate manufacturing process parameters that are scale-invariant. For instance, the manufacturing process parameter base total may be combined with total volume of the cell culture or VCD (e.g., by dividing the base total by the total volume of the cell culture or VCD, respectively) to create a new variable or manufacturing process parameter, base total per cell or base total per VCD, which can then be part of the reduced parameter set 108.

In various embodiments, the initial parameter set 106 may contain manufacturing process parameters that are measured over time (e.g., over the culture duration/time elapsed since the biomolecules manufacturing process is initiated). In such cases, the reduced training dataset 108 may include rates of these manufacturing process parameters computed by calculating the manufacturing process parameters over the culture duration. For example, the initial parameter set 106 may include lactate concentrations measured over a period of time (e.g., over the culture duration). In such cases, the reduced training dataset 108 may include a lactate concentration per time as a manufacturing process parameter. In some instances, the scale-invariant manufacturing process parameters (e.g., such as those expressed as functions of total volume of the cell culture or VCD) may also be expressed as a function of time (e.g., over the culture duration). For example, the initial parameter set 106 may include multiple measurements of the lactate concentration over the culture duration. In such cases, the lactate concentration over time may be combined with the total volume of the cell culture in the initial parameter set 106 to generate the new manufacturing process parameter, lactate concentration per cell per time, which can then be part of the reduced parameter set 108.

In various embodiments, the probabilistic graphical model 110 can be a Bayesian network model. A Bayesian network is an acyclic probabilistic graphical model that represents a set of random variables and their conditional independence with a directed acyclic graph (DAG). The Bayesian network could present the probabilistic relationship between one variable and another variable. Discussion related to the use of Bayesian network models in predicting glycosylation in cell culture media can be found in “Probabilistic model by Bayesian network for the prediction of antibody glycosylation in perfusion and fed-batch cell cultures,” by L. Zhang et al., which is incorporated by reference herein in its entirety. In various embodiments, the probabilistic graphical model 110 can be a Markov random field model.

Although the discussion herein pertains to the prediction of the glycan distribution using a probabilistic graphical model, in various embodiments, other computational methods can also be used for predicting the glycan distribution. For example, a machine learning model such as but not limited to a neural network, a decision tree, a random forest, a support vector machine (SVM), a regression tree, and/or the like, can be used to predict the glycan distribution. The neural network can be a deep neural network, a convolutional neural network (CNN), an artificial neural network (ANN), a recurrent neural network (RNN), a modular neural network (MNN), a residual neural network (RNN), an ordinary differential equations neural networks (neural-ODE), a squeeze and excitation embedded neural network, a MobileNet, a deep neural network, etc. The ANN can be a long short-term memory (LSTM) neural network. Decision tree learning models may include classification tree models, as well as regression tree models. The regression tree can be a gradient boosting machine (GBM) model (e.g., XGBoost). SVMs are a set of related supervised learning methods used for classification and regression. A SVM training algorithm—which may be a non-probabilistic binary linear classifier—may build a model that predicts whether a new example falls into one category or another. Other types of computational techniques are not discussed in detail herein for reasons of simplicity and it is understood that the present disclosure is not limited to a particular type of model.

In various embodiments, as discussed above, the probabilistic graphical model 110 can be trained with the reduced parameter set 108 to predict the glycan distribution 112. After training, the trained probabilistic graphical model 110 can be used to predict the distribution 112 of glycans that are attached to biomolecules produced by cell culture during a biomolecules manufacturing process, provided an input dataset 118 of manufacturing process parameters that are associated with that biomolecules manufacturing process is provided to the probabilistic graphical model 110. For example, one can measure or otherwise obtain the manufacturing process parameters of the initial parameter set 106 using sensors, probes, controllers, etc., operationally coupled to the bioreactor of the biomolecules manufacturing process, and these measurements can be compiled as the input dataset 118 of manufacturing process parameters. In some instances, this input dataset 118 can be pre-processed as discussed above to generate a reduced input dataset from which redundant, low-important, etc., manufacturing process parameters are removed. This reduced input dataset 118 may then be provided or input into the trained probabilistic graphical model 110 to predict the glycan distribution 112 of glycans attached to the cells during the biomolecules manufacturing process.

In various embodiments, the glycan distribution 112 as used herein refers to the relative proportions of glycans (e.g., carbohydrate structures) attached to the same location on a biomolecule. The glycan distribution 112 can be the end distribution of glycans at a location of a biomolecule after a glycoform evolves during the cell culture process from Man 5 glycan to other forms of glycans such as but not limited to G0F-N, G0-N, G0, G1, G0F, G1F, G2F, and/or the like. For example, for these eight glycans, the glycan distribution 112 can be the relative proportions (e.g., percentages) of these glycans that are attached to the same location of a biomolecule (e.g., an antibody) after a Man 5 glycan at that location evolves to one or more of the other seven glycans. It is to be noted that the above list of glycans is non-limiting and that the glycan distribution can refer to the relative proportion of any number of glycans that are attached to molecules at the same location. For example, glycan species with very low proportions in the glycan distribution may be grouped under a “dummy” glycan specie.

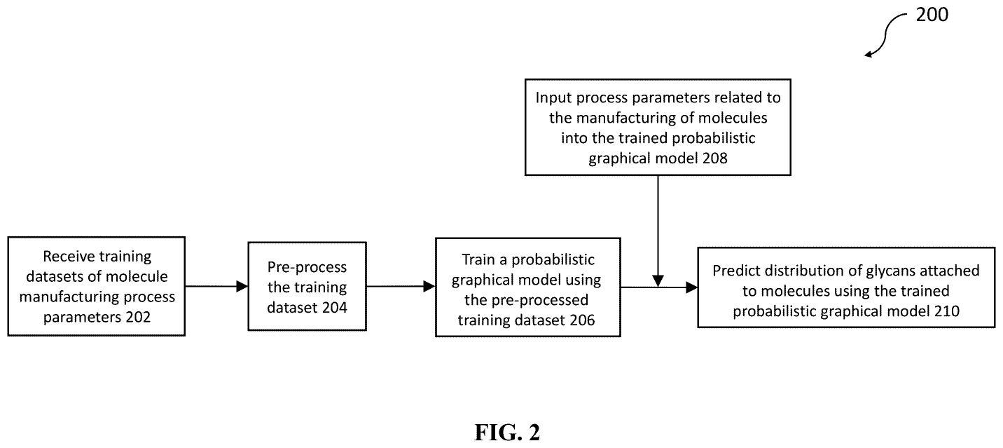

FIG. 2 is a workflow of a process 200 for training a probabilistic graphical model to predict distribution of glycans attached to molecules during a biomolecules manufacturing process, in accordance with various embodiments. In various embodiments, process 200 is implemented using a system, such as the glycan distribution prediction system 100 of FIG. 1, to predict the glycan distribution. For example, a technician may be tasked with producing molecules (e.g., monoclonal antibodies) in a cell culture in a bioreactor. In such cases, the technician may wish to determine the distribution of glycans attached to the molecules during the biomolecules manufacturing process (e.g., in real-time) because attached glycans can be used as quality attributes of the molecules. The technician may then utilize process 200 to train a probabilistic graphical model to predict glycan distributions and use the trained probabilistic graphical model to predict the distribution of glycans attached to the molecules being manufactured in the bioreactor (e.g., at a predetermine time (e.g., an end-point)). Such techniques are advantageous because they allow the technician to determine product quality of the manufacturing of the molecules without manual, and time- and resource-consuming sampling processes that may result in the contamination of the cell culture.

It should be appreciated that a manufacturing process parameter may refer to any component of a culture, including but not limited to serum components, nutrient components, waste components, biological cells, biological products, culture parameter(s) (that may be a molecular parameter, a cellular parameter, or a chemical parameter). In some embodiments, a culture parameter comprises any physicochemical or cellular characteristic of the culture including at least a nutrient, protein, peptide, amino acid, carbohydrate, growth factors, trace elements (e.g., cobalt, nickel, etc.), cytokine, salt, metal salt, fatty acids, lipids (e.g., cholesterol, steroids, and mixtures thereof), vitamins (group B vitamins, such as B12, vitamin A, vitamin E, riboflavin, thiamine, biotin, and mixtures thereof), the level of one or more constituents, the tonicity of a culture, the osmolality of a culture, the pH of a culture, the level of a cell in a culture, the total volume of the culture, and other similar parameters. When a nutrient is a parameter, the nutrient may comprise proteins, fats, carbohydrates (sugars, dietary fiber), vitamins, minerals, iron, sodium, mixtures thereof, and so forth. When a protein is a parameter, the protein may comprise antibodies, contractile proteins, enzymes, hormonal proteins, structural proteins, storage proteins, and/or transport proteins. When a carbohydrate is a parameter, the carbohydrate may include complex sugars and/or simple sugars, and may include glucose, maltose, fructose, galactose, and mixtures thereof. When an amino acid is a parameter, the amino acid may be glycine, alanine, valine, leucine, isoleucine, methionine, proline, phenylalanine, tryptophan, serine, threonine, asparagine, glutamine, tyrosine, cysteine, lysine, arginine, histidine, aspartic acid, glutamic acid, mixtures thereof, single stereoisomers thereof, racemic mixtures thereof and may also include non-standard amino acids, e.g., 4-hydroxyproline, ε-N,N,N-trimethyllysine, 3-methylhistidine, 5-hydroxylysine, O-phosphoserine, γ-carboxyglutamate, γ-N-acetyllysine, ω-N-methylarginine, N-acety !serine, N,N,N-trimethyalanine, N-formy !methionine, γ-aminobutyric acid, histamine, dopamine, thyroxine, citrulline, ornithine, b-cyanoalanine, homocysteine, azaserine, and S-adenosylmethionine.

In particular embodiments upon producing cell cultures, one aspect of the methods includes lowering product variability and increasing product quality by increasing control of the manufacturing process parameter(s) within the cell culture. One or more components of the process including the media may be maintained within predefined ranges by manipulating the manufacturing process parameter(s). In specific embodiments, one can systematically manipulate one or more process variables, such as media components and/or culture conditions, and observe the impact or ultimate outcome in the propagating cell culture. In specific embodiments, the minimum and maximum of a predefined range of a given manufacturing process parameter may be within a certain percentage of each other, such as within at least, or no more than, 0.05, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% or more of each other.

At step 202, a model training dataset of manufacturing process parameters may be received, for example at the glycan distribution prediction system 100, for use in training a probabilistic graphical model to predict the distribution of glycans attached to the molecules. In some instances, the training dataset may include records of manufacturing process parameters associated with the manufacturing of the molecules and associated glycan distributions of said molecules. For example, the records may be measurements of manufacturing process parameters and glycan distributions taken during previous manufacturing processes that were conducted to produce the molecules. For instance, the molecules that are to be manufactured may be monoclonal antibodies, and in such case, the training dataset may include records of manufacturing process parameters measured or otherwise obtained during prior manufacturing processes conducted to produce the monoclonal antibodies.

In various embodiments, the records of the training dataset may include the manufacturing process parameters of the initial parameter set 106 of FIG. 1. For example, the training dataset may include measurements of the following parameters of the biomolecules manufacturing process: amount of carbon dioxide sparged into the cell culture, amount of air sparged into the cell culture, amount of oxygen sparged into the cell culture, pH of the cell culture, base total, agitation control output, back pressure, total volume of the cell culture, pH output, temperature of the cell culture, the temperature of the bioreactor/vessel shell, temperature output of a bioreactor/vessel shell controller, culture duration, osmolality, glucose concentration, lactate concentration, sodium concentration, ammonium concentration, carbon dioxide concentration, oxygen concentration, conductivity of the cell culture, cell viability of the cells that produce the molecules, viable cell density (VCD) of the cells, packed cell volume (PCV) of the cells, immunoglobulin G (IgG) concentration, specific production of IgG (qIgG), cell growth rate, glucose uptake rate (GUR), and/or the like. In some instances, the training dataset can have additional, or less, manufacturing process parameters than listed here. Further, the training dataset may also include the distribution of glycans that were attached at the same location of the produced molecules. As such, the training dataset includes records of manufacturing process parameters of biomolecules manufacturing processes and associated glycan distributions of biomolecules manufacturing processes. In some instances, the manufacturing process parameters of the training dataset may be measured or obtained as described above with reference to the initial parameter set 106 of FIG. 1 (e.g., using sensors, probes, controllers, etc.).

At step 204, the training dataset may be pre-processed to, for example, remove manufacturing process parameters that have low contribution to the training of the probabilistic graphical model. For instance, all but one manufacturing process parameters of a subset of manufacturing process parameters can be redundant because the information that may be obtained from any one manufacturing process parameter of the subset and used for training the probabilistic graphical model is largely or entirely the same as that obtained from another manufacturing process parameter of the subset (e.g., and as such all but one may be discarded from the training dataset). As another example, a manufacturing process parameter may have low importance or contribution with respect to training the probabilistic graphical model to predict glycan distributions. For instance, the manufacturing process parameter may have little or no correlation with glycan distributions. In such cases, the training dataset may be pre-processed using one or more of the afore-discussed feature selection technique to discard the manufacturing process parameter from the training dataset. For example, the training dataset may be pre-processed using filter methods, wrapper methods, PCA, or combination thereof.

For example, filter methods utilizing Fisher score may be applied to the training dataset to rank the manufacturing process parameters in order of their importance or contribution to the training of the probabilistic graphical model. Further, filter methods utilizing correlation coefficients may be used to calculate the correlation coefficients between the manufacturing process parameters of the training dataset to identify redundancies in the training dataset. As another example, wrapper methods may be used to form one or more subsets of the manufacturing process parameters of the training dataset and evaluate the performance of the probabilistic graphical model in predicting the glycan distribution when these subsets are provided as input. In various embodiments, these methods may be applied to the training dataset to remove manufacturing process parameters that may be duplicative/redundant, low-importance (e.g., contribute little or none to the training of the probabilistic graphical model) and generate a reduced training dataset. It is to be understood are these are non-limiting examples and any feature selection methods can be applied to the training dataset of manufacturing process parameters to reduce the number of manufacturing process parameters therein and generate a reduced training dataset. After the pre-processing of the training dataset, in various embodiments, the reduced training dataset may include the remaining manufacturing process parameters and the glycan distributions of the (initial) training dataset.

In various embodiments, further to the pre-processing of the training dataset at step 204 as discussed above, the reduced training dataset may include the following manufacturing process parameters: amount of carbon dioxide sparged into the cell culture, amount of oxygen sparged into the cell culture, base total, total volume of the cell culture, culture duration, osmolality, lactate concentration, sodium concentration, cell viability, VCD, PCV, and qIgG. In some instances, the pre-processing may combine some of the manufacturing process parameters and may result in an eve more reduced training dataset. For example, in some instances, the manufacturing process parameters may be expressed as a function of time by combining (e.g., dividing) one or more manufacturing process parameters with the culture duration. As another example, these parameters may further be combined (e.g., divided by) the total volume of the cell culture and/or the VCD to be expressed as functions of cell culture volume or VCD. In such cases, the reduced training dataset may include one or more of the following manufacturing process parameters: amount of carbon dioxide sparged into the cell culture per VCD per time, amount of oxygen sparged into the cell culture per VCD per time, base total per VCD per time, osmolality per time, lactate concentration per cell culture volume per time, sodium concentration per cell culture volume per time, cell viability per time, PCV per time, and qIgG. It is to be understood that these are non-limiting examples and other combinations of the manufacturing process parameters are contemplated herein.

At step 206, in various embodiments, the reduced training dataset may be used to train the probabilistic graphical model to predict the distribution of glycans attached to the molecules during the biomolecules manufacturing process. That is, the manufacturing process parameters and the glycan distributions associated therewith in the reduced training dataset may be provided to the probabilistic graphical model to train the probabilistic graphical model to predict the glycan distributions based on an analysis of the provided manufacturing process parameters.

In various embodiments, once the probabilistic graphical model is trained, the effect of the manufacturing process parameters of the reduced training dataset on the prediction of the probabilistic graphical model may be computed. In some instances, the effect of a manufacturing process parameter on the prediction of the probabilistic graphical model may be quantified based on the correlation of the manufacturing process parameter with the predicted glycan distribution. For example, for the foregoing reduced training dataset, the effect of each of the manufacturing process parameters on the predicted glycan distribution may be determined by computing the correlation of the manufacturing process parameter with the predicted glycan distribution. In some instances, such computation may indicate the order of effect the manufacturing process parameters have on the predicted glycan distribution as discussed below.

In some instances, the correlation can be expressed by Pearson correlation coefficients relating the correlation between the manufacturing process parameters and the predicted glycan distribution. That is, the order of effect of the manufacturing process parameters on the glycan distribution that is predicted by the probabilistic graphical model can be established by ordering or ranking the Pearson correlation coefficients of each manufacturing process parameters with the glycan distribution. In some instances, the order of effect of the manufacturing process parameters on the glycan distribution may be generated using techniques or methods that provide explanations about the relationship between the features that are provided to models (e.g., the probabilistic graphical model) as inputs and the behavior or prediction of the models. For example, the technique permutation feature importance can be used to evaluate the importance of the manufacturing process parameters on the glycan distribution, and as such can be used to generate the order of effect of the manufacturing process parameters on the glycan distribution. Other techniques that can be used to generate the order of effect include partial dependence plots (PDP) or individual conditional expectation (ICE) plots that show the dependence between the manufacturing process parameters and the glycan distribution. Further, the order of effect can be generated using the technique Shapley Additive exPlanations (SHAP) that computes the contribution of each manufacturing process parameter to the prediction of the glycan distribution.

Once the probabilistic graphical model is trained to predict glycan distribution using the training dataset, in various embodiments, the trained probabilistic graphical model may be used to predict a glycan distribution of molecules that are manufactured in a cell culture in a bioreactor, provided measurements of the manufacturing process parameters of the bioreactor are provided to the trained probabilistic graphical model as input dataset. To do so, at step 208, manufacturing process parameters related to the biomolecules manufacturing process may be measured or otherwise obtained (e.g., using sensors, probes, controllers, a combination thereof, etc., or retrieved from databases) and at least some of them (e.g., at least three) may be provided to the trained probabilistic graphical model as input. In some instances, at step 210, the trained probabilistic graphical model may analyze the input data to predict the distribution of glycans that are attached to the molecules being produced by the biomolecules manufacturing process. For example, the trained probabilistic graphical model may generate percentages of glycans (e.g., percentages of one or more of Man 5, G0F-N, G0-N, G0, G1, G0F, G1F, or G2F) attached to the same location on the molecules that are being manufactured.

As example illustrations, the inventors of the instant disclosure have employed the workflow of process 200 of FIG. 2 to train probabilistic graphical models to predict distribution of glycans that are attached to the same location on molecules that are produced by a biomolecules manufacturing process. In a first embodiment, a probabilistic graphical model was trained using a reduced training dataset that includes the following manufacturing process parameters: amount of carbon dioxide sparged into the cell culture per VCD per time, amount of oxygen sparged into the cell culture per VCD per time, base total per VCD per time, osmolality per time, lactate concentration per cell culture volume per time, sodium concentration per cell culture volume per time, cell viability per time, PCV per time, and qIgG.

In various embodiments, the order of effect of these manufacturing process parameters on the glycan distribution was generated as discussed above. The manufacturing process parameters listed in order of effect on the glycan distribution are shown in Table 1, where Table 1 is as follows:

| Serial | |
|---|---|
| No. | Manufacturing process parameter |
| 1 | lactate concentration per cell culture volume per time |
| 2 | osmolality per time |
| 3 | base total per VCD per time |
| 4 | cell viability per time |
| 5 | sodium concentration per cell culture volume per time |
| 6 | amount of oxygen sparged into the cell culture per VCD per time |
| 7 | amount of carbon dioxide sparged into the cell culture per VCD |
| | per time |
| 8 | PCV per time |
| 9 | qIgG |

In a second embodiment, a probabilistic graphical model was trained using a reduced training dataset that includes the following manufacturing process parameters: amount of carbon dioxide sparged into the cell culture, amount of oxygen sparged into the cell culture, base total, total volume of the cell culture, culture duration, osmolality, lactate concentration, sodium concentration, cell viability, VCD, PCV, and qIgG.

In Table 1, “Serial No.” is an index denoting the order of effect of the corresponding manufacturing process parameter indexed by that serial number on the glycan distribution that is predicted by the probabilistic graphical model.

In various embodiments, after a probabilistic graphical model is trained using a training dataset, the trained probabilistic graphical model may be used to predict the distribution of glycans attached to molecules that are produced by a biomolecules manufacturing process. In some instances, the manufacturing process parameters (e.g., listed in Table 1) of the biomolecules manufacturing process may be measured or otherwise obtained. In such cases, a selected number of the measured manufacturing process parameters may then be provided to the trained probabilistic graphical model for analysis so the probabilistic graphical model can predict the distribution of glycans attached to the molecules. For Table 1, the selected number can be at least three, at least four, at least five, at least six, at least seven, at least eight, or all nine of the manufacturing process parameters. In some instances, the number of input manufacturing process parameters can depend on the desired level of accuracy of the trained probabilistic graphical model's prediction of the glycan distribution.

In some embodiments, any training dataset for any machine learning model may comprise data from a parameter consisting of, consisting essentially of, or comprising lactate concentration per cell culture volume per time. In some embodiments, any training dataset may comprise data from a parameter consisting of, consisting essentially of, or comprising osmolality per time. In some embodiments, any training dataset may comprise data from a parameter consisting of, consisting essentially of, or comprising base total per VCD per time. In some embodiments, any training dataset may comprise data from a parameter consisting of, consisting essentially of, or comprising cell viability per time. In some embodiments, any training dataset may comprise data from a parameter consisting of, consisting essentially of, or comprising sodium concentration per cell culture volume per time. In some embodiments, any training dataset may comprise data from a parameter consisting of, consisting essentially of, or comprising amount of oxygen sparged into the cell culture per VCD per time. In some embodiments, any training dataset may comprise data from a parameter consisting of, consisting essentially of, or comprising amount of carbon dioxide sparged into the cell culture per VCD per time. In some embodiments, any training dataset may comprise data from a parameter consisting of, consisting essentially of, or comprising PCV per time. In some embodiments, any training dataset may comprise data from a parameter consisting of, consisting essentially of, or comprising qIgG.

In various embodiments, one or multiple manufacturing process parameters may be analyzed for a trained machine learning model to generate an indicator of glycan distribution on biomolecules. In specific embodiments, a manufacturing process parameter comprises at least lactate concentration per cell culture volume per time, or a manufacturing process parameter does not comprise lactate concentration per cell culture volume per time. In specific embodiments, a manufacturing process parameter comprises at least osmolality per time, or a manufacturing process parameter does not comprise osmolality per time. In specific embodiments, a manufacturing process parameter comprises at least base total per VCD per time, or a manufacturing process parameter does not comprise base total per VCD per time. In specific embodiments, a manufacturing process parameter comprises at least cell viability per time, or a manufacturing process parameter does not comprise cell viability per time. In specific embodiments, a manufacturing process parameter comprises at least sodium concentration per cell culture volume per time, or a manufacturing process parameter does not comprise sodium concentration per cell culture volume per time. In specific embodiments, a manufacturing process parameter comprises at least amount of oxygen sparged into the cell culture per VCD per time, or a manufacturing process parameter does not comprise amount of oxygen sparged into the cell culture per VCD per time. In specific embodiments, a manufacturing process parameter comprises at least amount of carbon dioxide sparged into the cell culture per VCD per time, or a manufacturing process parameter does not comprise amount of carbon dioxide sparged into the cell culture per VCD per time. In specific embodiments, a manufacturing process parameter comprises at least PCV per time, or a manufacturing process parameter does not comprise PCV per time. In specific embodiments, a manufacturing process parameter comprises at least qIgG, or a manufacturing process parameter does not comprise qIgG.

In various embodiments, a training data set for a machine learning model comprises, consists of, or consists essentially of 1, 2, 3, 4, 5, 6, 7, 8, or 9 of any of the following: lactate concentration per cell culture volume per time; osmolality per time; base total per VCD per time; cell viability per time; sodium concentration per cell culture volume per time; amount of oxygen sparged into the cell culture per VCD per time; amount of carbon dioxide sparged into the cell culture per VCD per time; PCV per time; and qIgG. As discussed above, the use of predictive models such as probabilistic graphical models to determine glycan distributions without necessarily having to obtain cell culture samples can be advantageous because of increased manufacturing efficiencies as well as reduced or eliminated sample contamination risks. Further, if there is a relationship between a manufacturing process parameter and a glycan, then the prediction of the glycan distribution using predictive models may allow one to adjust in real-time the manufacturing process parameter so as to arrive at a desired glycan distribution (e.g., a glycan distribution that indicates superior quality attribute for the molecules being produced by the manufacturing process). For example, a probabilistic graphical model may predict a glycan distribution for a biomolecules manufacturing process (e.g., producing molecules such as monoclonal antibodies, etc.), and the glycan distribution may indicate lower proportion of G2F (e.g., further indicating low quality of molecules). In such cases, if it is known that a manufacturing process parameter (e.g., amount of oxygen in the cell culture) is known to have a strong correlation with G2F glycans, then more oxygen may be sparged into the cell culture until the probabilistic graphical model predicts that glycan distribution indicates an at least adequate G2F proportion (e.g., and as such at least adequate quality of molecules being produced by the biomolecules manufacturing process).

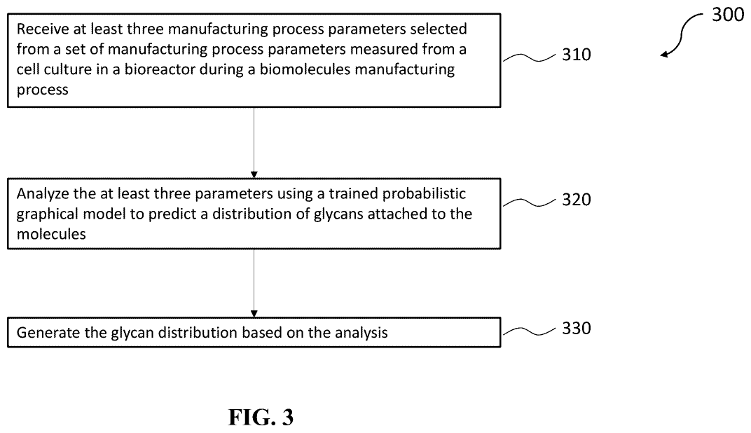

FIG. 3 shows a flowchart of a method 300 for predicting distribution of one or more glycans attached to molecules during a biomolecules manufacturing process, in accordance with various embodiments. In various embodiments, method 300 is implemented using a system, such as, the glycan distribution prediction system 100 of FIG. 1.

At step 302, at least three manufacturing process parameters selected from a set of manufacturing process parameters are received at a processor. In some instances, the set of manufacturing process parameters are measured from a cell culture in a bioreactor during the biomolecules manufacturing process, wherein each manufacturing process parameter of the set of manufacturing process parameters is listed in Table 1 in order of effect on the glycan distribution. Examples of molecules produced by the biomolecules manufacturing process of method 300 include monoclonal antibodies.

At step 304, the at least three manufacturing process parameters are analyzed using the trained probabilistic graphical model to predict the glycan distribution.

At step 306, the trained probabilistic graphical model generates the glycan distribution based on the analysis.

In various embodiments of method 300, the probabilistic graphical model can be a Bayesian network model or a Markov random field model.

In various embodiments, the glycan distribution indicates relative proportions of the one or more glycans attached to the molecules. Examples of the one or more glycans include Man5, G0F-N, G0-N, G0, G1, G0F, G1F, or G2F. In various embodiments, method 300 further comprises adjusting the one of the at least three manufacturing process parameters to change the relative proportions of the one or more glycans.

In various embodiments of method 300, at least one of the set of manufacturing process parameters of Table 1 is measured by a sensor operationally connected to the bioreactor. For example, the at least one of the set of manufacturing process parameters is the total volume of the cell culture or the osmolality of the cell culture, and the sensor is a scale configured to weigh the cell culture or an osmometer, respectively, disposed within the bioreactor.

In various embodiments of method 300, the at least one of the set of manufacturing process parameters is an output of a controller operationally connected to the bioreactor. For example, the one of the set of manufacturing process parameters is the amount of carbon dioxide sparged into the cell culture, or the amount of oxygen sparged into the cell culture, and the controller is an air flow controller configured to control flow of the carbon dioxide sparged into the cell culture or the oxygen sparged into the cell culture, respectively.

### IV. Pre-Processing of Training Dataset

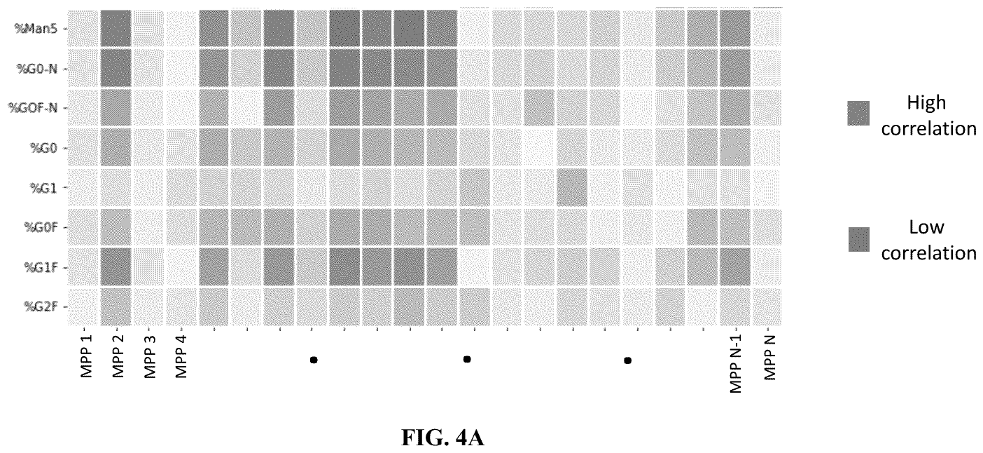

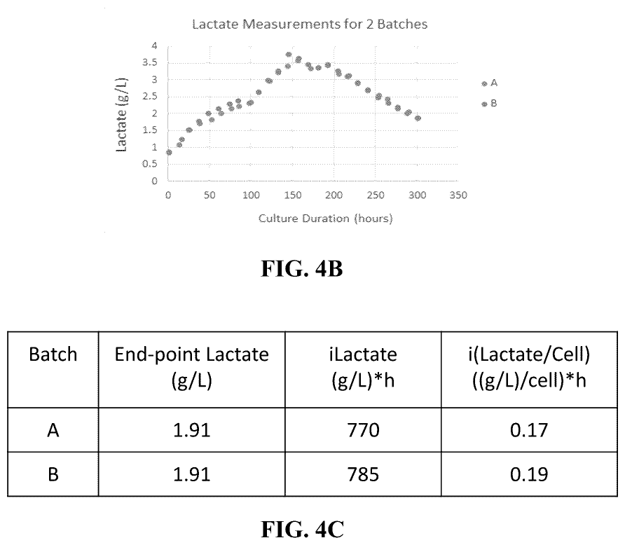

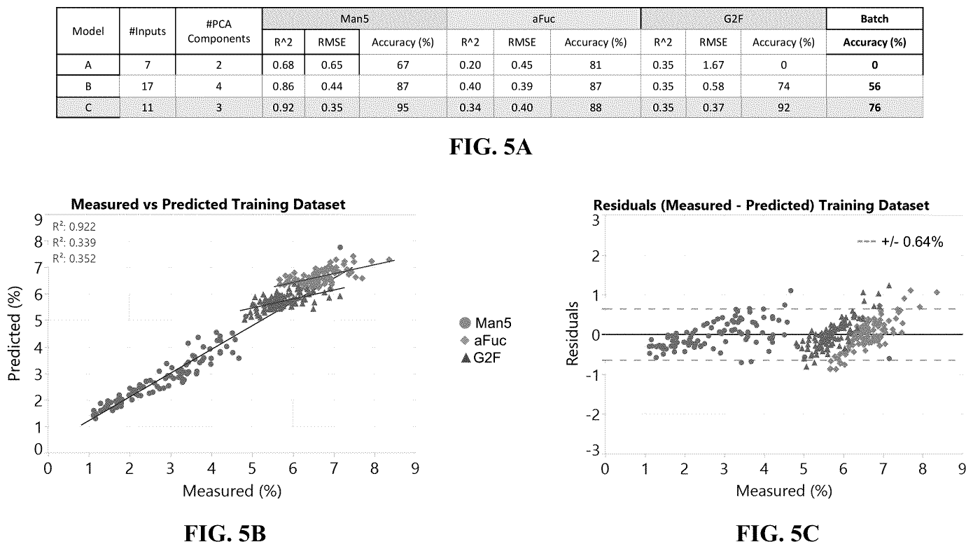

FIG. 4A shows a heatmap (FIG. 4A), a graph (FIG. 4B) and a table (FIG. 4C) illustrating the pre-processing of a training dataset of manufacturing process parameters for use in training a probabilistic graphical model to predict distribution of glycans attached to molecules during a biomolecules manufacturing process, in accordance with various embodiments. In various embodiments, as discussed above, an initial set of manufacturing process parameters related to a process for manufacturing molecules in a bioreactor may be measured (e.g., using probes, sensors, controllers, etc.) or otherwise obtained (e.g., retrieved from a database) to include into a training dataset for training the probabilistic graphical model. The manufacturing process parameters include but are not limited to an amount of carbon dioxide sparged into the cell culture, an amount of air sparged into the cell culture, an amount of oxygen sparged into the cell culture, a pH of the cell culture, a concentration of dO₂ in the bioreactor, the base total, the agitation control output, the back pressure, the total volume of the cell culture, the temperature of the cell culture, the jacket temperature, the temperature output of the bioreactor/vessel shell controller, the culture duration, the glucose concentration, the lactate concentration, the sodium concentration, the ammonium concentration, the osmolality, the PCV, the CO₂ concentration, the O₂ concentration, the conductivity of the cell culture, the cell viability, the VCD, the PCV, the concentration of IgG, the qIgG, the cell growth rate, the GUR, and/or the like.

In some instances, one may wish to reduce this initial set of manufacturing process parameters to remove those manufacturing process parameters that have little or no contribution towards the training the probabilistic graphical model. In such cases, the importance of the manufacturing process parameters of the initial set with respect to the training of the probabilistic graphical model may be computed, and the initial set narrowed down to a reduced set of manufacturing process parameters based on the computed measure of importance. For instance, the manufacturing process parameters having associated therewith an importance score below a threshold importance score may be discarded and not included in the training dataset (i.e., the initial training dataset may be reduced).

In some instances, the importance of the manufacturing process parameters may be computed using a feature selection technique. For example, the afore-mentioned filter methods may be applied to the manufacturing process parameters of the initial set to quantify the importance of the manufacturing process parameters for training the probabilistic graphical model. For instance, Fisher scores of the manufacturing process parameters may be computed to quantify the importance of the manufacturing process parameters for training for training the probabilistic graphical model. As another example, Pearson correlation coefficients of the manufacturing process parameters with output of the probabilistic graphical model (e.g., glycan distribution) may also be used to quantify the importance of the manufacturing process parameters for training the probabilistic graphical model. Statistical techniques utilizing information gain/mutual information, variance threshold, etc., may also be used to determine the importance of the manufacturing process parameters.

In various embodiments, at least some of the manufacturing process parameters of the initial set may have little correlation with most or all of the glycan species, and in such cases, these manufacturing process parameters may be removed from the training dataset based on the correlation values. For example, when computing the distribution of Man 5, G0F-N, G0-N, G0, G1, G0F, G1F, or G2F attached to molecules during a biomolecules manufacturing process, the correlation of each of the manufacturing process parameters with the proportions of each glycan may be computed. Then, the manufacturing process parameters found to have low correlation with the proportions of most or all of the glycans may be discarded from the training dataset. FIG. 4A shows an example heatmap illustrating the correlations between a set of manufacturing process parameters (MPP) and the proportions of the afore-listed eight glycans. In such instances, manufacturing process parameters such as MPP 4 that have low correlation with all or almost all of the glycans may not be included in the training dataset.

In various embodiments, the correlation matrix between the manufacturing process parameters and the proportions of glycan in the glycan distribution (e.g., such as the one shown in FIG. 4A) may also be used in determining which manufacturing process parameter to adjust to arrive at a desired glycan distribution. As discussed above, glycans (and distribution thereof) can be quality attributes of the molecules being produced by the biomolecules manufacturing process. If a glycan distribution indicates that the molecules have poor quality and that a particular glycan has low production (e.g., low proportion), then one can consult the correlation matrix to identify the manufacturing process parameter that is strongly correlated with that particular glycan. In such case, the manufacturing process parameter can be adjusted (e.g., increased) to increase the production of the particular glycan (e.g., and consequently increase the quality of the molecules). For instance, with reference to FIG. 4A, if a glycan distribution indicates that G1F formation is low (e.g., and the molecules being produced have low quality), then one can adjust MPP N−1 (e.g., or any of the other MPPs strongly correlated with the proportion of G1F) to increase the proportion of G1F (e.g., and consequently the quality of the molecules).

FIG. 4B shows a plot of example lactate concentration measurements taken during the culture durations of two molecules manufacturing batch processes, in various embodiments. Glycans can be used as end-point quality attributes of the production of molecules by biomolecules manufacturing processes. However, more context or information about the manufacturing processes may be obtained by combining two or more of the manufacturing process parameters. For example, the lactate concentration and the culture duration (e.g., time elapsed since initiation of the batch manufacturing processes) can be combined to determine the rate of lactate concentrations. FIGS. 4B-4C indicate that although the end-point lactate concentrations are the same for batches A and B, the rate of lactate concentrations are not the same during the batch manufacturing processes, providing context and additional information about batches A and B. The rates of lactate concentrations may also be combined with other manufacturing process parameters, for instance with VCD determine the lactate concentration per time per cell manufacturing process parameters, which may provide additional information about the batch manufacturing processes (e.g., as shown in FIG. 4C).

### V. Training and Testing of Probabilistic Graphical Model

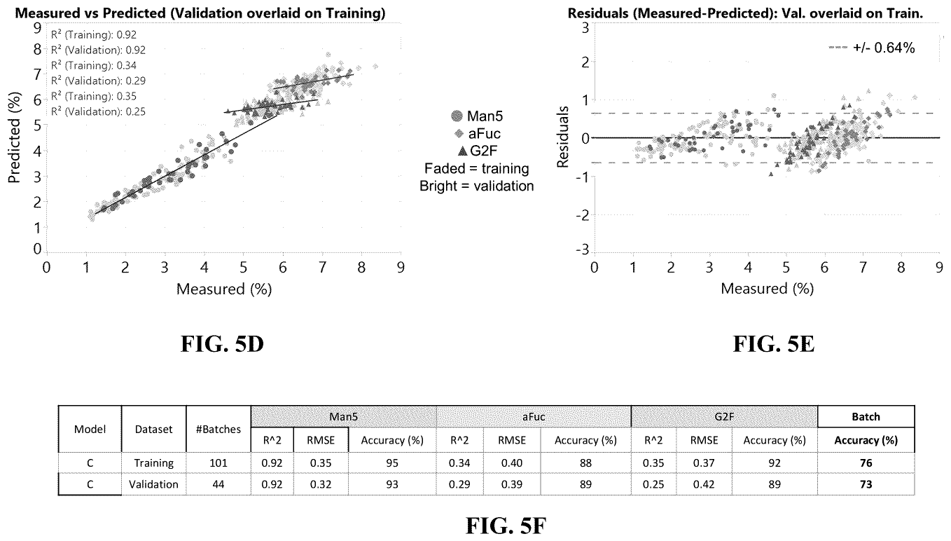

FIGS. 5A-5F show illustrations of the training and testing of a probabilistic graphical model to predict the distribution of glycans attached to molecules during a biomolecules manufacturing process, in accordance with various embodiments. The probabilistic graphical model was trained and used to predict the glycan distribution with respect to the glycans Man 5, G2F and non-fucosylated (“aFuc”) that are attached to the same location on the molecules. It is to be to understood that the choice of these glycan species is solely for illustrative purposes, and that the probabilistic graphical model can be trained and used to predict the distribution of any species of glycans.

The probabilistic graphical model was trained using three sets of training datasets, referred in FIG. 5A as “Model A”, “Model B”, and Model C”. Model A denotes the probabilistic graphical model that is trained with a training dataset of seven manufacturing parameters to predict the glycan distribution, i.e., the relative proportions of Man 5, G2F, and aFuc that are attached to molecules being produced by a biomolecules manufacturing process. A principal component analysis was performed to linearly combine the seven manufacturing process parameters to construct two principal components. The training of the probabilistic graphical model includes computing the accuracy of the model's predictions of the glycan distribution (e.g., with respect to offline assay measurements of proportions of the glycan species). FIG. 5A shows that the trained probabilistic graphical model has very low accuracy, in particular with its prediction of the proportion of G2F glycans.

Model B denotes the probabilistic graphical model that is trained with a training dataset of seventeen manufacturing parameters to predict the relative proportions of Man 5, G2F, and aFuc that are attached to the molecules. A principal component analysis was performed to linearly combine the seventeen manufacturing process parameters to construct four principal components. The training of the probabilistic graphical model includes computing the accuracy of the model's predictions of the glycan distribution (e.g., with respect to offline assay measurements of proportions of the glycan species). FIG. 5A shows that the trained probabilistic graphical model has improved accuracy than Model A.

Model C denotes the probabilistic graphical model that is trained with a training dataset of eleven manufacturing parameters to predict the relative proportions of Man 5, G2F, and aFuc that are attached to the molecules. A principal component analysis was performed to linearly combine the eleven manufacturing process parameters to construct three principal components. The training of the probabilistic graphical model includes computing the accuracy of the model's predictions of the glycan distribution (e.g., with respect to offline assay measurements of proportions of the glycan species). FIG. 5A shows that model C has the best accuracy of the three considered probabilistic graphical models. FIGS. 5B and 5C show plots illustrating the accuracy of Model C's prediction of the relative proportions of Man 5, G2F, and aFuc with respect to the measured proportions.

The training of Model C included obtaining measurements of manufacturing process parameters and glycan distributions from about 145 batches of biomolecules manufacturing processes. The batches were split randomly into training (70% of the batches ˜101 batches) datasets and validation datasets (30% of the batches ˜44 batches). The validation dataset is used to provide an unbiased test of the fit of the model. FIGS. 5D-5F show that the glycan distribution prediction results of Model C when the training dataset is provided to the model as training input are close to the respective results of Model C when the validation dataset (e.g., which is independent of the training dataset) is provided to the model as a testing input, confirming that model C is capable of accurately predicting distributions of glycans that are attached to molecules during a biomolecules manufacturing process.

### VI. Computer Implemented System

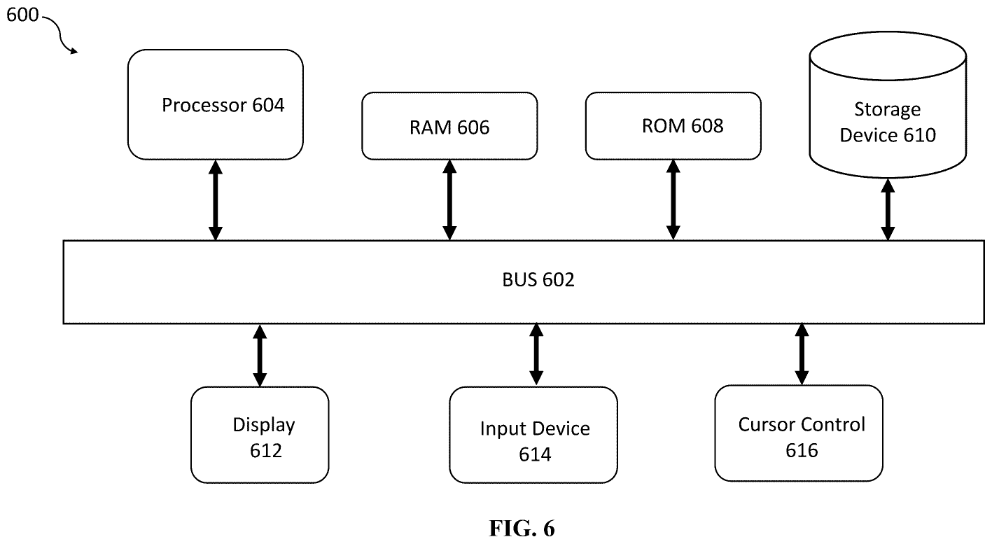

FIG. 6 is a block diagram of a computer system in accordance with various embodiments. Computer system 600 may be an example of one implementation for glycan distribution prediction system 100 described above in FIG. 1. In one or more examples, computer system 600 can include a bus 602 or other communication mechanism for communicating information, and a processor 604 coupled with bus 602 for processing information. In various embodiments, computer system 600 can also include a memory, which can be a random-access memory (RAM) 606 or other dynamic storage device, coupled to bus 602 for determining instructions to be executed by processor 604. Memory also can be used for storing temporary variables or other intermediate information during execution of instructions to be executed by processor 604. In various embodiments, computer system 600 can further include a read only memory (ROM) 608 or other static storage device coupled to bus 602 for storing static information and instructions for processor 604. A storage device 610, such as a magnetic disk or optical disk, can be provided and coupled to bus 602 for storing information and instructions.

In various embodiments, computer system 600 can be coupled via bus 602 to a display 612, such as a cathode ray tube (CRT) or liquid crystal display (LCD), for displaying information to a computer user. An input device 614, including alphanumeric and other keys, can be coupled to bus 602 for communicating information and command selections to processor 604. Another type of user input device is a cursor control 616, such as a mouse, a joystick, a trackball, a gesture input device, a gaze-based input device, or cursor direction keys for communicating direction information and command selections to processor 604 and for controlling cursor movement on display 612. This input device 614 typically has two degrees of freedom in two axes, a first axis (e.g., x) and a second axis (e.g., y), that allows the device to specify positions in a plane. However, it should be understood that input devices 614 allowing for three-dimensional (e.g., x, y and z) cursor movement are also contemplated herein.

Consistent with certain implementations of the present teachings, results can be provided by computer system 600 in response to processor 604 executing one or more sequences of one or more instructions contained in RAM 606. Such instructions can be read into RAM 606 from another computer-readable medium or computer-readable storage medium, such as storage device 610. Execution of the sequences of instructions contained in RAM 606 can cause processor 604 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 604 for execution. Such a medium can take many forms, including but not limited to, non-volatile media, volatile media, and transmission media. Examples of non-volatile media can include, but are not limited to, optical, solid state, magnetic disks, such as storage device 610. Examples of volatile media can include, but are not limited to, dynamic memory, such as RAM 606. Examples of transmission media can include, but are not limited to, coaxial cables, copper wire, and fiber optics, including the wires that comprise bus 602.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 604 of computer system 600 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 600 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 600, whereby processor 604 would execute the analyses and determinations provided by these engines, subject to instructions provided by any one of, or a combination of, the memory components RAM 606, ROM, 608, or storage device 610 and user input provided via input device 614.

### VII. Glycans and Biomolecules Comprising Same

In various embodiments, the present disclosure concerns prediction of distribution of glycans on biomolecules manufactured in a cell culture. The glycans on the molecules may be of any kind. The glycans may be O-linked or N-linked. The glycans may be plant, animal, or microbial glycans. In particular embodiments, the source of the glycans is from eukaryotic cells of any kind.

In particular embodiments, the biomolecules on which the glycans may be located may be proteins or lipids. In specific embodiments, the glycans are distributed on antibodies of any kind, including monoclonal antibodies.

In some embodiments, a polypeptide (such as an antibody) expressed by the cells of the present disclosure may bind to, or interact with, any protein, including, without limitation, cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, ILIRAP, ILIRAPL1, ILIRAPL2, IL1RN, IL6ST, IL18BP, IL18RAP, IL22RA2, AIF1, HGF, LEP (leptin), PTN, and THPO.k.

In some embodiments, a polypeptide (such as an antibody) expressed by the cells of the present disclosure may bind to, or interact with, a chemokine, chemokine receptor, or a chemokine-related protein selected from the group consisting of CCLI (1-309), CCL2 (MCP-1/MCAF), CCL3 (MIP-Iα), CCL4 (MIP-Iβ), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL. In some embodiments, the polypeptide expressed by the host cells of the present disclosure may bind to, or interact with, 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL 11 (eotaxin); CCL13 (MCP-4); CCL15 (MIP18); CCL16 (HCC-4); CCL17 (TARC); CCL18 (PARC); CCL19 (MIP-3β); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5 (RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79α, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL1l (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDGl; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-α; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; 1L13; IL13RA1; IL13RA2; 1114; 1115; IL15RA; 1L16; 1L17; IL17B; IL17C; IL17R; 1118; IL18BP; IL18R1; I18RAP; IL19; IL IA; IL1B; ILIF10; IL1F5; IL1F6; IL1F7; IL1F8; IL1F9; IL1HY1; IL1R1; IL1R2; ILIRAP; IL1RAPL1; ILIRAPL2; IL1RL1; IL1RL2, IL1RN; 112; 1120; IL20Rα; IL21 R; IL22; IL-22c; IL22R; IL22RA2; IL23; IL24; 1L25; 1126; 1127; IL28A; IL28B; 1L29; 1L2RA; 1L2RB; 1L2RG; 113; IL30; 1L3RA; 1L4; IL4R; 115; IL5RA; 116; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67, RIG-E, SCA-2, TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NRID1; NRID2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; DI2S53E; PMEL17; SI; SIL); PPBP (CXCL7); PPID; PRI; PRKCQ; PRKDI; PRL; PROC; PROK2; PSAP; PSCA hlg (2700050C12Rik, C530008O16Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B); SERPINA1; SERPINA3; SERPINB5 (maspin); SERPINE1 (PAI-1); SERPDMF1; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-1b); XCRI (GPR5/CCXCRI); YY1; and/or ZFPM2.

In certain embodiments, target molecules for antibodies (or bispecific antibodies) produced according to the methods disclosed herein include CD proteins such as CD3, CD4, CDS, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD79β, IGb (immunoglobulin-associated beta), B29); CD200 members of the ErbB receptor family such as the EGF receptor, HER2, HER3, or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc. In certain embodiments, the methods provided herein can be used to produce an antibody (or a multispecific antibody, such as a bispecific antibody) that specifically binds to complement protein C5 (e.g., an anti-C5 agonist antibody that specifically binds to human C5).

### VIII. Recitation of Various Embodiments of the Present Disclosure

Embodiment 1: A computer-implemented method for predicting a glycan distribution of one or more glycans attached to molecules during a biomolecules manufacturing process, the method comprising: receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution; analyzing, via the processor, the at least three parameters using a trained probabilistic graphical model to predict the glycan distribution; and generating, via the processor, the glycan distribution based on the analyzing.

Embodiment 2: The method of embodiment 1, wherein the probabilistic graphical model is a Bayesian network model.

Embodiment 3: The method of embodiment 1 or 2, wherein the probabilistic graphical model is a Markov random field model.

Embodiment 4: The method of any of embodiments 1-3, wherein: the glycan distribution indicates relative proportions of the one or more glycans attached to the molecules, the method further comprising adjusting the one of the at least three manufacturing process parameters to change the relative proportions of the one or more glycans.

Embodiment 5: The method of any of embodiments 1-4, wherein the glycans include one or more of Man5, G0F-N, G0-N, G0, G1, G0F, G1F, or G2F.

Embodiment 6: The method of any of claims 1-5, wherein at least one of the set of manufacturing process parameters is measured by a sensor operationally connected to the bioreactor.

Embodiment 7: The method of embodiment 6, wherein the at least one of the set of manufacturing process parameters is the total volume of the cell culture or the osmolality of the cell culture, and the sensor is a scale configured to weigh the cell culture or an osmometer, respectively, disposed within the bioreactor.

Embodiment 8: The method of any of embodiments 1-5, wherein at least one of the set of manufacturing process parameters is an output of a controller operationally connected to the bioreactor.

Embodiment 9: The method of embodiment 8, wherein the one of the set of manufacturing process parameters is the amount of carbon dioxide sparged into the cell culture, or the amount of oxygen sparged into the cell culture, and the controller is an air flow controller configured to control flow of the carbon dioxide sparged into the cell culture or the oxygen sparged into the cell culture, respectively.

Embodiment 10: The method of any of embodiments 1-9, wherein the molecules include a monoclonal antibody.

Embodiment 11. A system for predicting a glycan distribution of one or more glycans attached to molecules manufactured in a cell culture in a bioreactor, the system comprising: a non-transitory memory storing instructions; and a processor coupled to the non-transitory memory and configured to read the instructions from the non-transitory memory to cause the system to perform any of the methods of embodiments 1-10.

Embodiment 12. A non-transitory computer-readable medium (CRM) having stored thereon computer-readable instructions executable to cause performance of operations for predicting a glycan distribution of one or more glycans attached to molecules manufactured in a cell culture in a bioreactor, the operations comprising any of the methods of embodiments 1-10.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A computer-implemented method for predicting a glycan distribution of one or more glycans attached to molecules during a biomolecules manufacturing process, the method comprising:
receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution;
generating, via the processor, an indicator of glycan distribution by providing the at least three parameters as input to a probabilistic graphical model that has been trained to predict the glycan distribution using training data comprising, for each of a plurality of cell cultures in a biomolecules manufacturing process, values of the manufacturing process parameters and corresponding measured values of the indicator of glycan distribution.

2. The method of claim 1, wherein the probabilistic graphical model is a Bayesian network model and/or is a Markov random field model.

3. (canceled)

4. The method of claim 1, wherein the glycan distribution indicates relative proportions of the one or more glycans attached to the molecules, the method further comprising:
adjusting at least one of the set of manufacturing process parameters to change the relative proportions of the one or more glycans.

5. The method of claim 1, wherein the glycans include one or more of Man5, G0F-N, G0-N, G0, G1, G0F, G1F, or G2F.

6. The method of claim 1, wherein at least one of the set of manufacturing process parameters is measured by a sensor operationally connected to the bioreactor or wherein at least one of the set of manufacturing process parameters is an output of a controller operationally connected to the bioreactor.

7. The method of claim 6, wherein the at least one of the set of manufacturing process parameters is the total volume of the cell culture or the osmolality, and the sensor is a scale configured to weigh the cell culture or an osmometer, respectively, disposed within the bioreactor.

8. (canceled)

9. The method of claim 6, wherein the one of the set of manufacturing process parameters is the amount of carbon dioxide sparged into the cell culture, or the amount of oxygen sparged into the cell culture, and the controller is an air flow controller configured to control flow of the carbon dioxide sparged into the cell culture or the oxygen sparged into the cell culture, respectively.

10. The method of claim 1, wherein the molecules include a monoclonal antibody.

11. A system for predicting a glycan distribution of one or more glycans attached to molecules during a biomolecules manufacturing process, the system comprising:
a non-transitory memory storing instructions; and
a processor coupled to the non-transitory memory and configured to read the instructions from the non-transitory memory to cause the system to perform operations comprising:
receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution; and
analyzing the at least three parameters using a trained probabilistic graphical model to predict the glycan distribution; and
generating the glycan distribution based on the analyzing.

12. The system of claim 11, wherein the probabilistic graphical model is a Bayesian network model and/or is a Markov random field model.

13. (canceled)

14. The system of claim 1, wherein the glycan distribution indicates relative proportions of the one or more glycans attached to the molecules, the operations further comprising:
adjusting at least one of the set of manufacturing process parameters to change the relative proportions of the one or more glycans.

15. The system of claim 11, wherein the glycans include one or more of Man5, G0F-N, G0-N, G0, G1, G0F, G1F, or G2F.

16. A non-transitory computer-readable medium (CRM) having stored thereon computer-readable instructions executable to cause performance of operations for predicting a glycan distribution of one or more glycans attached to molecules during a biomolecules manufacturing process, the operations comprising:
receiving, at a processor, at least three manufacturing process parameters selected from a set of manufacturing process parameters measured from a cell culture in a bioreactor during the biomolecules manufacturing process, wherein each manufacturing process parameter of the set of manufacturing process parameters is listed in Table 1 in order of effect on the glycan distribution;
analyzing the at least three parameters using a trained probabilistic graphical model to predict the glycan distribution; and
generating the glycan distribution based on the analyzing.

17. The non-transitory CRM of claim 16, wherein the probabilistic graphical model is a Bayesian network model and/or wherein the probabilistic graphical model is a Markov random field model.

18. (canceled)

19. The non-transitory CRM of claim 16, wherein the glycan distribution indicates relative proportions of the one or more glycans attached to the molecules, the operations further comprising:
adjusting at least one of the set of manufacturing process parameters to change the relative proportions of the one or more glycans.

20. The non-transitory CRM of claim 16, wherein the glycans include one or more of Man5, G0F-N, G0-N, G0, G1, G0F, G1F, or G2F.

21. A computer-implemented method for predicting a glycan distribution of one or more glycans attached to molecules during a biomolecules manufacturing process, the method comprising:
receiving, at a processor, at least three manufacturing process parameters selected from a set of manufacturing process parameters measured from a cell culture in a bioreactor during the biomolecules manufacturing process, wherein the at least three manufacturing process parameters are selected from the group consisting of lactate concentration per cell culture volume per time; osmolality per time; base total per VCD per time; cell viability per time; sodium concentration per cell culture volume per time; amount of oxygen sparged into the cell culture per VCD per time; amount of carbon dioxide sparged into the cell culture per VCD per time; PCV per time; and qIgG;
generating an indicator of glycan distribution, via the processor, by providing the at least three parameters as input to a probabilistic graphical model that has been trained to predict the glycan distribution using training data comprising, for each of a plurality of cell cultures in a biomolecule manufacturing process, values of the at least three manufacturing process parameters and corresponding measured values of the indicator of glycan distribution.
